(19) United States
(12) Patent Application Publication
(10) Pub. No.: US 2003/0228279 A1
(43) Pub. Date: Dec. 11, 2003

(54) Compositions and methods for viral delivery

(75) Inventors: Sally Mossman, Seattle, WA (US); Lawrence Evans, Seattle, WA (US); Ryan M. Swanson, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corixa Corporation, Seattle, WA

(21) Appl. No.: 10/283,484

(22) Filed: Oct. 29, 2002

Related U.S. Application Data
(60) Provisional application No. 60/369,715, filed on Apr. 3, 2002.
(60) Provisional application No. 60/335,512, filed on Oct. 31, 2001.

(51) Int. Cl. A61K 48/00; A61K 38/20; A61K 38/19; A61K 31/739

(52) U.S. Cl. 424/93.2; 514/54; 424/85.1; 424/85.2; 536/53

(57) ABSTRACT

Compositions and methods comprising a recombinant virus and an immunostimulant are provided for enhancing the immune response to a polypeptide expressed from the recombinant virus. Preferably this is done without also enhancing the neutralizing antibody response to the recombinant virus. Illustrative compositions comprise an adenovirus and an adjuvant such as, for example, monophosphoryl lipid A, an alkyl glucosaminide phosphate, a saponin, or a combination thereof. The disclosed compositions and methods are useful, for example, in the treatment of diseases such as cancer or infectious disease.

### CROSS-REFERENCE TO RELATED APPLICATIONS

This application claims the priority of U.S. Provisional Application Serial No. 60/335,512, filed Oct. 31, 2002, and No. 60/369,715, filed Apr. 3, 2002, the disclosures of which are hereby incorporated herein in their entirety.

### BACKGROUND OF THE INVENTION

1. Technical Field of the Invention

The present invention relates generally to compositions and methods for viral delivery and, more specifically, to compositions and methods comprising combinations of recombinant viruses and immunostimulants, such as adjuvants, having improved immunological properties.

2. Description of the Related Art

Conventional compositions and methodologies employing recombinant viruses in combination with immunostimulants such as, for example, Seppic adjuvant ISA206 for delivery and protein expression of therapeutic polynucleotides are frequently hampered by the in vivo induction of neutralizing antibody responses that effectively blocks viral efficacy. See, e.g., Adam et al., Veterinary Microbiology 42:205-215 (1994). Thus, there remains a need in the art for improved compositions and methods that permit immunization regimens employing reduced recombinant viral titers while maintaining strong immune responses. As described in further detail herein, the compositions and methods of the present invention fulfill this need and further provide other related advantages.

### SUMMARY OF THE INVENTION

In one aspect, the present invention provides compositions and methods employing a recombinant virus and one or more immunostimulants, such as one or more adjuvants. Within certain embodiments, the recombinant virus may be selected from the group consisting of an adenovirus, an adeno-associated virus (AAV), a pox virus, and an alphavirus. Alternative embodiments provide that the immunostimulant is an adjuvant selected from the group consisting of Freund's Incomplete Adjuvant; Freund's Complete Adjuvant; Merck Adjuvant 65; AS-2; aluminum hydroxide gel; aluminum phosphate; a salt of calcium, iron or zinc; an insoluble suspension of acylated tyrosine acylated sugars; cationically or anionically derivatized polysaccharides; polyphosphazenes; biodegradable microspheres; aminoalkyl glucosaminide phosphates; monophosphoryl lipid A compounds; saponins and mineral oil/water adjuvants. Preferred embodiments provide compositions and methods employing one or more adenorecombinant viruses in combination with the adjuvant monophosphoryl lipid A and/or derivatives thereof.

In certain embodiments, the compositions of the present invention are immunogenic, i.e., they are capable of eliciting an immune response, particularly a humoral and/or cellular immune response, as further described herein. Preferably, inventive compositions result in improved efficacy of the recombinant virus while permitting immunization with lower viral dose and consequent reduction in neutralizing antibody response.

According to methods of the present invention, the combination of virus and adjuvant may be administered in a prime and/or boost regimen.

Within other aspects, the present invention provides pharmaceutical compositions comprising one or more recombinant virus in combination with one or more immunostimulants, such as an adjuvant, as described above, further in combination with a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and one or more immunostimulants, such as an adjuvant.

Compositions according to the present invention may employ recombinant viruses carrying one or more polynucleotides that encode one or more polypeptide antigens including proteins and/or fusion proteins. Such compositions may be in the form of pharmaceutical compositions, e.g., vaccine compositions, comprising a physiologically acceptable carrier in addition to one or more immunostimulant, such as an adjuvant. The fusion proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with a cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with a cancer, in which case the methods provide treatment for the disease, or patient considered at risk for such a disease may be treated prophylactically.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 is a graph depicting average cytotoxic T-lymphocyte (CTL) activity following subcutaneous (SC) or intradermal (ID) immunization of mice with adenovirus alone or in combination with MPL-AF (aqueous formulation of monophosphoryl lipid A) or MPL-SE (oil emulsion of monophosphoryl lipid A).

FIG. 2 is a chart depicting interferon-gamma (IFN-γ) response to recombinant tuberculosis antigen (rTbH9) from mice immunized with TbH9 adenovirus alone or in combination with ISA206 [a water in (mineral) oil in water adjuvant available from Seppic, France], MPL-AF or MPL-SE.

FIG. 3 is a chart depicting anti-adenovirus ELISA and neutralizing antibody titers following subcutaneous (SC) or intradermal (ID) immunization of mice with adenovirus alone or in combination with ISA206, MPL-AF or MPL-SE.

FIG. 4 is a chart depicting ICC staining after stimulation with El in pooled splenocytes from mice immunized with adenovirus alone or in combination with, MPL-AF or MPL-SE.

FIG. 5 shows the enhanced CD4 and CD8 immune response to the TbH9 antigen.

FIG. 6 shows percentage of CD8 cells secreting interferon-gamma (IFN-γ) in response to re-stimulation with EL-4-TbH9 cells and concentration of interferon-gamma (ng/mL) in splenocytes supernatant stimulated with rTbH9.

FIG. 7 is a chart depicting anti-adenovirus ELISA and neutralizing antibody titers following intramuscular immunization of mice with adenovirus alone or in combination with MPL-AF, B19-TEoA, B20-TEoA, B15-TEoA, B-38-TEoA, and B-39-TEoA. “TEoA” as used herein stands for triethanolamine. The “B” designations refer to specific adjuvant compounds whose structures are described below.

### DETAILED DESCRIPTION OF THE INVENTION

The present invention is directed generally to compositions and methods for immunization of a mammal. Compositions according to the present invention employ one or more recombinant virus such as, for example, an adenovirus in combination with an immunostimulant, such as an adjuvant. Inventive compositions and methods permit immunization regimens employing reduced viral titers while retaining and/or improving upon a strong immune response. Such compositions and methods, as discussed in further detail herein below, may be employed in the therapy of cancer and/or infectious disease.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984). All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Recombinant Viruses

As described above, compositions and methods according to the present invention employ one or more recombinant virus in combination with one or more immunostimulant, such as an adjuvant, in order to achieve the desired immunological properties, preferably while also minimizing or eliminating the elicitation of neutralizing antibodies, while enhancing the immune response directed against a polypeptide expressed from the recombinant virus.

In order to express a desired polypeptide, the nucleotide sequences encoding the polypeptide, or functional equivalents, may be inserted into a recombinant virus that contains the necessary elements for the transcription and translation of the inserted coding sequence. Methods, known to those skilled in the art, may be used to construct recombinant viruses containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

The “control elements” or “regulatory sequences” present in a viral expression vector are those non-translated regions of the vector—enhancers, promoters, 5′ and 3′ untranslated regions—which interact with viral and/or host cellular proteins to carry out transcription and translation. Such elements may vary in their strength and specificity. Promoters from mammalian genes or from mammalian viruses are generally preferred.

In mammalian host cells, a number of viral-based expression systems are generally available that may be suitable for the compositions and methods of the present invention. For example, in the specific case where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Bums et al. (1993) Proc. Natl. Acad. Sci. USA 90:8033-8037; and Boris-Lawrie and Temin (1993) Cur. Opin. Genet. Develop. 3:102-109.

A number of illustrative adenovirus-based systems have been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional recombinant viruses useful for delivering polypeptides of the present invention include those derived from the pox family of viruses, such as vaccinia virus and avian poxvirus. By way of example, vaccinia virus recombinants can be constructed as follows. The polynucleotide encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

Immunostimulants

In addition to the recombinant virus systems described herein above, compositions and methods of the present invention further comprise one or more immunostimulants. As used herein, the term immunostimulant refers generally to a substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortaclella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A, certain saponins and certain mineral oil/water adjuvants such as the ISA series (Seppic, France). Cytokines, such as GM-CSF, interleukin-2, -7, -12, and other like growth factors, may also be used as adjuvants.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell-mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Preferred adjuvants for eliciting a predominantly Th1-type response include, for example, a combination of a monophosphoryl lipid A, preferably 3-de-O-acylated monophosphoryl lipid A, together with an aluminum salt. MPL® adjuvants that contain monophosphoryl acid A are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996.

Some preferred adjuvants in the compositions and methods of this invention include aminoalkyl glucosaminide phosphate (AGP) compounds and saponin compounds, as described in more detail below.

Aminoalkyl Glucosaminide Phosphates (AGPs)

Aminoalkyl glucosaminide phosphate (AGP) compounds generally comprise a 2-deoxy-2-amino-α-D-glucopyranose (glucosaminide) in glycosidic linkage with a cyclic or acyclic aminoalkyl (aglycon) group. Suitable AGP compounds, and methods for their synthesis and use, are described generally in U.S. Pat. Nos. 6,113,918 and 6,303,347, WO 98/50399, U.S. patent application Ser. No. 09/074,720 filed May 7, 1998, International patent application PCT/US01/24284, and Johnson et al. (1999) Bioorg. Med. Chem. Lett. 9: 2273-2278, the disclosures of which are incorporated herein by reference in their entirety.

AGP compounds employed in the compositions of the present invention may be monosaccharide or disaccharide compounds. Thus, the present invention provides compositions that comprise one or more AGP compounds having the formula:

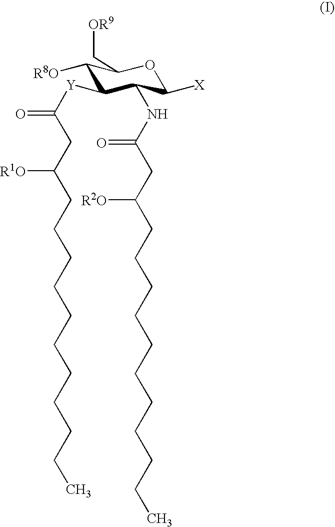

and pharmaceutically acceptable salts and derivatives thereof, wherein Y is —S—, —O— or —NH—; R¹ and R² are each independently selected from saturated and unsaturated (C₂-C₂₄) aliphatic acyl groups; R⁸ is —H or —PO₃R¹¹R¹², wherein R¹¹ and R¹² are each independently —H or (C₁-C₄) aliphatic groups; R⁹ is —H, —CH3 or —PO₃R¹³R¹⁴, wherein R¹³ and R¹⁴ are each independently selected from —H and (C₁-C₄) aliphatic groups; and wherein at least one of R⁸ and R⁹ is a phosphorus-containing group, but R⁸ and R⁹ are not both phosphorus-containing groups; and X is a group selected from the formulae:

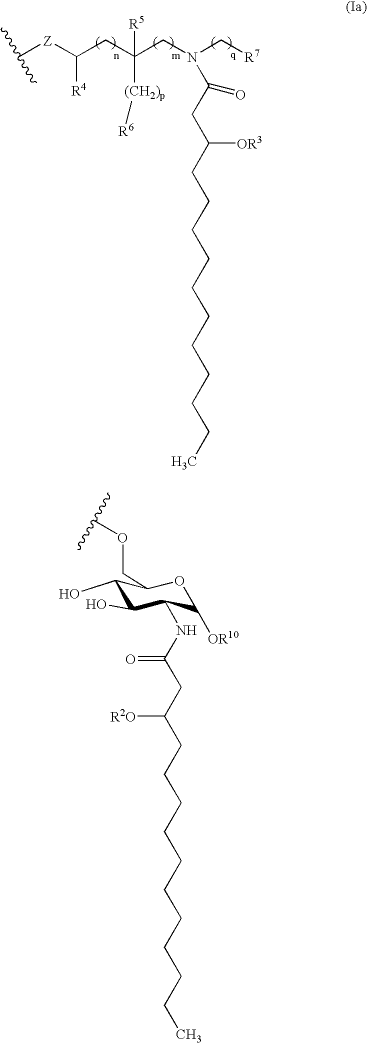

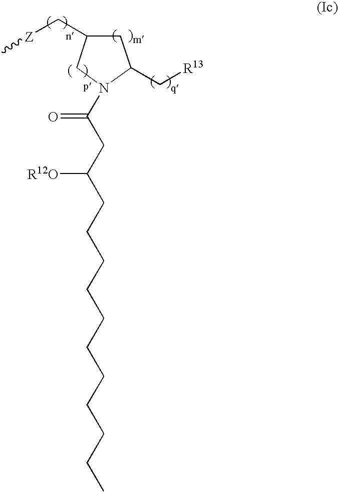

wherein the subscripts n, m, p, q, n′, m′, p′ and q′ are each independently an integer of from 0 to 6, provided that the sum of p′ and m′ is an integer from 0 to 6; R³, R¹¹, and R¹² are independently a saturated or unsaturated optionally substituted aliphatic (C₂-C₂₄) acyl group, provided that when X is formula (Ia), one of R¹, R² and R³ is optionally hydrogen; R⁴ and R⁵ are independently selected from H and methyl; R⁶ and R⁷ are independently selected from H, OH, (C₁-C₄) oxyaliphatic groups, —PO₃H₂, —OPO₃H₂, —SO₃H, —OSO₃H, —NR¹⁵R¹⁶, —SR¹⁵, —CN, —NO₂, —CHO, —CO₂R¹⁵, —CONR¹⁵R¹⁶, —PO3R¹⁵R¹⁶, —OPO₃R¹⁵R¹⁶, —SO₃R¹⁵ and —OSO₃R⁵, wherein R¹⁵ and R¹⁶ are each independently selected from H and (C₁-C₄) aliphatic groups; R¹⁰ is selected from H, CH₃, —PO₃H₂, ω-phosphonooxy(C₂-C₂₄)alkyl, and ω-carboxy(C₁-C₂₄)alkyl; R¹³ is independently selected from H, OH, (C₁-C₄) oxyaliphatic groups, —PO₃R⁷R¹⁸, —OPO₃R¹⁷R¹⁸, —SO₃R¹⁷, —OSO₃R¹⁷, —NR¹⁷R¹⁸, —SR¹⁷, —CN, —NO₂, —CHO, —CO₂R¹⁷, and —CONR¹⁷R¹⁸, wherein R¹⁷ and R¹⁸ are each independently selected from H and (C₁-C₄) aliphatic groups; and Z is —O— or —S—.

One type of AGP compound of the present invention can be described generally by the following structure:

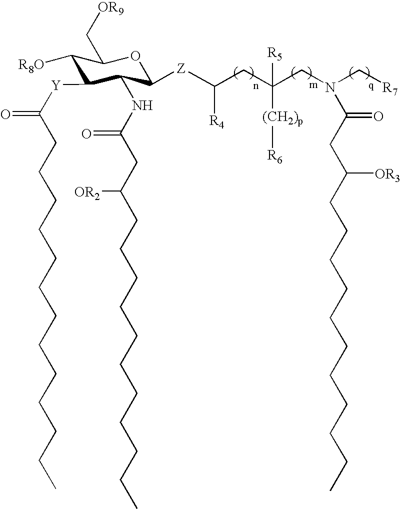

and pharmaceutically acceptable salts, derivatives and biologically active fragments thereof, wherein Z represents an oxygen or sulfur atom, Y represents an oxygen atom or NH group, “n”, “m”, “p” and “q” are integers independently selected from 0 to 6, R₁, R₂ and R₃ represent fatty acyl residues, including saturated, unsaturated, and branched acyl groups, having 6 to 16 carbon atoms, R₄ and R₅ are independently selected from hydrogen and methyl, R₆ and R₇ are independently selected from hydrogen, hydroxy, alkoxy, phosphono, phosphonooxy, sulfo, sulfooxy, amino, mercapto, cyano, nitro, formyl or carboxy and esters and amides thereof; R₈ and R₉ are independently selected from phosphono or hydrogen, wherein at least one of R₈ and R₉ is phosphono. The configuration of the 3′ stereogenic centers to which the normal fatty acyl residues are attached is R or S, but preferably R. The stereochemistry of the carbon atoms to which R₄ or R₅ are attached can be R or S. All stereoisomers, both enantiomers and diastereomers, and mixtures thereof, are considered to fall within the scope of the present invention. See, U.S. Pat. No. 6,113,918.

Alternatively, AGP compounds employed in the immunostimulant compositions may generally conform to the following structure:

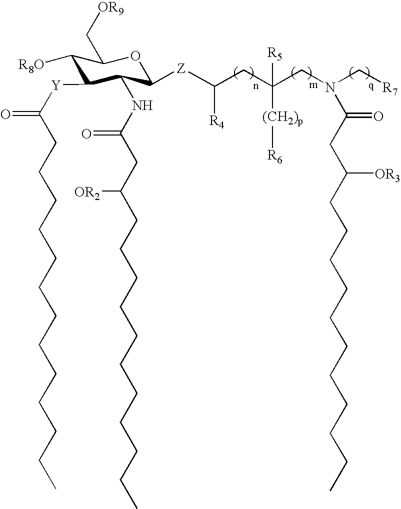

and pharmaceutically acceptable salts, derivatives and biologically active fragments thereof, wherein Z represents an oxygen or sulfur atom in either the axial or equatorial position; Y represents an oxygen atom or NH group; “n”, “m”, “p” and “q” are integers independently selected from 0 to 6; R₁, R₂, and R₃ represent fatty acyl residues, including saturated, unsaturated, and branched acyl groups, having 1 to 20 carbon atoms and where one of R₁, R₂ or R₃ is optionally hydrogen; R₄ and R₅ are independently selected from hydrogen or methyl; R₆ and R₇ are independently selected from hydrogen, hydroxy, alkoxy, phosphono, phosphonooxy, sulfo, sulfooxy, amino, mercapto, cyano, nitro, formyl or carboxy and esters and amides thereof; R₈ and R₉ are independently selected from phosphono or hydrogen, wherein at least one of R₈ and R₉ is phosphono. See, U.S. Pat. No. 6,303,347.

Still further AGP compounds generally conform to the following structure:

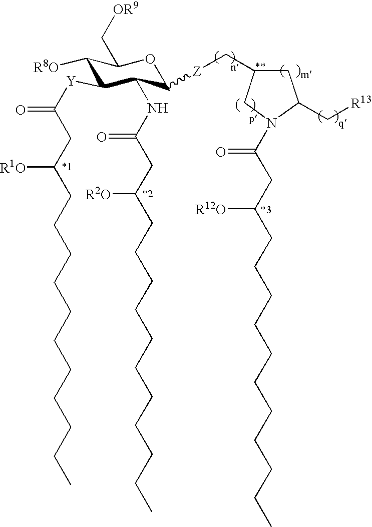

and pharmaceutically acceptable salts thereof, wherein Z is a member selected from the group consisting of —O— and —NH—; Y is a member selected from the group consisting of —O— and —S—; R¹, R² and R³ are each members independently selected from the group consisting of (C₂-C₂₄) acyl; R⁴ is a member selected from the group consisting of —H and —PO₃R⁷R⁸, wherein R⁷ and R⁸ are each members independently selected from the group consisting of —H and (C₁-C₄)alkyl; R⁵ is a member selected from the group consisting of —H, —CH₃ and —PO₃R⁹R¹⁰, wherein R⁹ and R¹⁰ are each members independently selected from the group consisting of —H and (C₁-C₄)alkyl; R⁶ is selected from H, OH, (C₁-C₄)alkoxy, —PO₃R¹¹R¹², —OPO₃R¹¹R², —SO₃R¹¹, —OSO₃R¹¹, —NR¹¹R¹², —SR¹¹, —CN, —NO₂, —CHO, —CO₂R¹¹, and —CONR¹¹R¹², wherein R¹¹ and R¹² are each independently selected from H and (C₁-C₄)alkyl, with the provisos that one of R⁴ and R⁵ is a phosphorus-containing group and that when R⁴ is —PO₃R⁷R⁸, R⁵ is other than —PO₃R⁹R¹⁰; wherein “*1”, “*2”, “*3” and “**” represent chiral centers; wherein the subscripts n′, m′, p′ and q′ are each independently an integer from 0 to 6, with the proviso that the sum of p′ and m′ is from 0 to 6. See PCT application WO 02/12258. Within certain embodiments, R¹, R² and R³ are each members independently selected from the group consisting of (C₉-C₁₆) acyl, or from the group consisting of (C₁₀-C₁₄) acyl, or from the group consisting of (C₁₀-C₁₂) acyl. The heteroatoms Y and Z of the AGP compounds can be oxygen or sulfur or —NH, as indicated. In a preferred embodiment, Z is oxygen and typically in the equatorial position. Although the stability of the molecules could be affected by a substitution at Z, the immunomodulating activity of molecules with these substitutions is not expected to change.

The number of carbon atoms between heteroatom Z and the aglycon nitrogen atom is determined by variables “n” and “m” for compounds (la) and n′ and m′ for compounds where X is formula (1c). Variables “n”, “m”, n′ and m′ can be integers from 0 to 6. In a preferred embodiment for compounds where X is formula (1a), the total number of carbon atoms between heteroatom Z and the aglycon nitrogen atom is from about 2 to about 6 and most preferably from about 2 to about 4. In such compounds, “n” and “m” are preferably both 0.

The AGPs are phosphorylated, such as at position 4 or 6 (formula Ia, R₈ or R₉) on the glucosaminide ring. For example, in one illustrative AGP of formula (Ia), R₈ is phosphono and R₉ is hydrogen. In one embodiment, the AGPs are hexaacylated that is they contain a total of six fatty acid residues or acyl groups. The aminoalkyl glucosaminide moiety is acylated at the 2-amino and 3-hydroxyl groups of the glucosaminide unit and at the amino group of the aglycon unit with 3-hydroxyalkanoyl residues. In Formula (Ia), these three positions are acylated with 3-hydroxytetradecanoyl moieties. The 3-hydroxytetradecanoyl residues are, in turn, substituted with normal fatty acids (R₁-R₃), providing three 3-n-alkanoyloxytetradecanoyl residues or six fatty acid groups in total.

In another embodiment, the AGP compounds are pentaacylated, that is, they contain a total of five fatty acid residues or acyl groups. More specifically, the 3-hydroxytetradecanoyl residues of Formula (Ia) are substituted with normal acyl groups at two of the three R₁, R₂ and R₃ positions, with the third R₁, R₂ or R₃ position being hydrogen. In other words, at least one of —OR₁, —OR₂ or —OR₃ is hydroxyl.

The chain length of normal fatty acid residues or acyl groups R₁-R₃ in the AGPs can be from 2 to about 24. Preferably, R₁-R₃ have chain lengths of from about 6 to about 16 carbons, more preferably from about 6 to about 12 carbons, and most preferably from about 6 to about 10 carbons. Within these ranges, the chain lengths of these normal fatty acid residues can be the same or different. Although, only normal fatty acids are described, it is expected that unsaturated fatty acids (i.e. fatty acid moieties having double or triple bonds) substituted at R₁-R₃ on the compounds of the present invention would produce biologically active molecules. Further, slight modifications in the chain length of the 3-hydroxyalkanoyl residues are not expected to dramatically affect biological activity.

Preferred embodiments of the invention include compositions containing AGP compounds as defined above and methods of use of such compositions, having one or more of the following:

R¹, R², R³, R¹¹ and R¹² preferably are (C₆-C₁₆) aliphatic acyl groups, even more preferably (C₆-C₁₂) aliphatic acyl groups, and most preferably (C₆-C₁₀) aliphatic acyl groups;

R⁶ is COOH;

X is formula (Ia) and R¹, R², and R³ are all acyl groups (i.e., the compounds are hexa-acylated);

X is formula (Ia) and one of R¹, R² and R³ is hydrogen (i.e. the compounds are penta-acylated);

Z is oxygen;

when R⁸ or R⁹ is a phosphorus-containing group, such group preferably is an unsubstituted phosphoro group (R¹¹ and R¹², or R¹³ and R¹⁴, respectively, are both hydrogen); more preferably R⁸ is a phosphorus-containing group and R⁹ is hydrogen;

the total of n+m is an integer from 0 to 4, preferably 0, 1 or 2;

n and m are both 0;

p and q are independently 0, 1 or 2;

n and p are both 0;

n′, m′, p′ and q′ are preferably independently an integer from 0 to 3; more preferably 0, 1, or 2; and most preferably n′ is 1, m′ is 2 and p′ and q′ are both 0 [i.e., the compounds of this type, where Y is formula (Ic), have a 2-pyrrolidinylmethyl configuration]

Especially preferred compounds are those in which X is formula (Ia), R¹, R² and R³ are all acyl groups, and n and p are both 0 or in which X is formula (Ia), R¹, R² and R³ are all acyl groups and R⁶ is COOH. Within both of these types of compounds, R¹, R² and R³ are preferably the same or different (C₆-C₁₆) aliphatic acyl groups, even more preferably the same or different (C₆-C₁₂) aliphatic acyl groups, and most preferably are the same or different (C₆-C₁₀) aliphatic acyl groups.

Another type of AGP usable in compositions of this invention is monophosphoryl lipid A (MLA). MLA is described in U.S. Pat. Nos. 4,436,727; 4,877,611; 4,866,034 and 4,912,094; 4,987,237; Johnson et al., J Med Chem 42:4640-4649 (1999); Ulrich and Myers, in Vaccine Design: The Subunit and Adjuvant Approach; Powell and Newman, Eds.; Plenum: N.Y., 495-524, 1995; the disclosures of which are incorporated herein by reference in their entireties. MLA often is in the form of a mixture of compounds that contains a mixture of disaccharides, some of which are of the formula (Ib), and some of which have a structure similar to formula (Ib) but have lesser degrees of acylation.

The MLA product sold under the trademark MPL® is the 3-O-desacyl-4′-monophosphoryl lipid A (3D-MLA) obtained by sequential acid and base hydrolyses of the lipopolysaccharide (LPS) from the R595 strain of S. minnesota. MPL® is a mixture of closely related monophosphoryl lipid A (MLA) species (congeners) that all contain the same backbone, consisting of a β-1′,6-linked disaccharide of 2-deoxy-2-aminoglucose that is phosphorylated at the 4′ position, but that differ in the fatty acid substitutions at the 2, 2′ and 3′ positions. The 1, 3, and 6′ positions of the backbone are unsubstituted in all MLA species present in MPL®. The 2, 2′ and 3′ positions may be substituted with tetradecanoic, 3-(R)-hydroxytetradecanoic, or 3-(R)-acyloxytetradecanoic acids, depending on the position, such that the total number of fatty acyl groups varies from three to six. The 3-(R)-acyloxytetradecanoic acid moieties contain primarily dodecanoic, tetradecanoic or hexadecanoic acids, depending on the backbone position.

The following are illustrative subtypes of AGP compounds of formula (Ia).

In one illustrative class of such AGPs, R₆ is carboxy, Z is O; Y is O; n, m, p and q are 0; R₁, R₂ and R₃ are normal fatty acyl residues having 10 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is carboxy, Z is O; Y is O; n, m, p and q are 0; R₁, R₂ and R₃ are normal fatty acyl residues having 12 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is carboxy, Z is O; Y is O; n, m, p and q are 0; R₁, R₂ and R₃ are normal fatty acyl residues having 10 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an R configuration.

In another illustrative class of such AGPs, R₆ is carboxy, Z is O; Y is O; n, m, p and q are 0; R₁, R₂ and R₃ are normal fatty acyl residues having 8 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is H, Z is O; Y is O; n is 2; m, p and q are 0; R₁, R₂ and R₃ are normal fatty acyl residues having 14 carbon atoms; R₄, R₅ and R₇ are H; R₈ is phosphono; R₉ is H; and R₁, R₂ and R₃ are each attached to a stereogenic center having an R configuration.

In another illustrative class of such AGPs, R₆ is H, Z is O; Y is O; n is 1, m and p are 0; q is 1; R₁, R₂ and R₃ are normal fatty acyl residues having 10 carbon atoms; R₄ and R₅ are H; R₇ is carboxy; R₈ is phosphono; R₉ is H; and R₁, R₂ and R₃ are each attached to a stereogenic center having an R configuration.

In another illustrative class of such AGPs, R₆ is H, Z is O; Y is O; m, n, p and q are 0; R₁, R₂ and R₃ are normal fatty acyl residues having 14 carbon atoms; R₄, R₅ and R₇ are H; R₈ is phosphono; R₉ is H; and R₁, R₂ and R₃ are each attached to a stereogenic center having an R configuration.

In another illustrative class of such AGPs, R₆ is H, Z is O; Y is O; m, n, p and q are 0; R₁, R₂ and R₃ are normal fatty acyl residues having 10 carbon atoms; R₄, R₅ and R₇ are H; R₈ is phosphono; R₉ is H; and R₁, R₂ and R₃ are each attached to a stereogenic center having an R configuration.

In another illustrative class of such AGPs, R₆ is H, Z is O; Y is O; m, p and q are 0; n is 1; R₁, R₂ and R₃ are normal fatty acyl residues having 14 carbons; R₄, R₅ and R₇ are H; R₈ is phosphono; R₉ is H; and R₁, R₂ and R₃ are each attached to a stereogenic center having an R configuration.

In another illustrative class of such AGPs, R₆ is hydroxy, Z is O; Y is O; m, n and q are 0; p is 1; R₁, R₂ and R₃ are normal fatty acyl residues having 12 carbon atoms; R₄ and R₅ are H; R₇ is H; R₈ is phosphono; and R₉ is H; R₁, R₂ and R₃ are each attached to a stereogenic center having an R configuration; and R₅ is attached to a stereogenic center having an S configuration.

In another illustrative class of such AGPs, R₆ is hydroxy, Z is O; Y is O; m and q are 0; n and p are 1; R₁, R₂ and R₃ are normal fatty acyl residues having 10 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is hydroxy, Z is O; Y is O; m, n and q are 0; p is 2; R₁, R₂ and R₃ are normal fatty acyl residues having 10 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is hydroxy, Z is O; Y is O; m, n and q are 0; p is 1; R₁, R₂ and R₃ are normal fatty acyl residues having 14 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an R configuration.

In another illustrative class of such AGPs, R₆ is hydroxy, Z is O; Y is O; m, n and q are 0; p is 1; R₁, R₂ and R₃ are normal fatty acyl residues having 14 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is hydroxy, Z is O; Y is O; m, n and q are 0; p is 1; R₁, R₂ and R₃ are normal fatty acyl residues having 11 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is hydroxy, Z is O; Y is O; m, n and q are 0; p is 1; R₁, R₂ and R₃ are normal fatty acyl residues having 10 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, Z is O; Y is O; m, n, p and q are 0; R₁, R₂ and R₃ are normal fatty acyl residues having 10 carbon atoms; R₄ and R₅ are H; R₆ is amino carbonyl; R₇ is H; R₈ is phosphono; and R₉ is H; R₁, R₂ and R₃ are each attached to a stereogenic center having an R configuration; and R₅ is attached to a stereogenic center having an S configuration.

In one particularly preferred embodiment of the invention, the AGP is 2-[(R)-3-Tetradecanoyloxytetradecanoylamino]ethyl 2-Deoxy-4-O-phosphono-3-O-[(R)-3-tetradecanoyloxytetradecanoyl]-2-[(R)-3-tetradecanoyloxytetradecanoylamino]-O-D-glucopyranoside triethylammonium salt. This corresponds to a compound having the structure set forth in Formula (Ia) in which R₁═R₂═R₃=n-C₁₃H₂₇CO, Z=Y═O, n=m=p=q=0, R₄═R₅═R₆═R₇═R₉=H, and R₈═PO₃H₂, and is referred to herein as compound B-19.

In additional embodiments of the invention, preferred AGP compounds of Formula (Ia) include the following:

| Ref. No. | R₁-R₃ | n | p | R₆ | q | R₇ |
|---|---|---|---|---|---|---|
| B2** | n-C₁₃H₂₇CO | 0 | 1 | OH | 0 | H |
| B3 | n-C₁₁H₂₃CO | 0 | 1 | OH | 0 | H |
| B9 | n-C₉H₁₉CO | 1 | 1 | OH | 0 | H |
| B14** | n-C₉H₁₉CO | 0 | 0 | CO₂H | 0 | H |
| B15* | n-C₉H₁₉CO | 0 | 0 | CO₂H | 0 | H |
| B20 | n-C₉H₁₉CO | 0 | 0 | H | 0 | H |
| B21 | n-C₁₃H₂₇CO | 1 | 0 | H | 0 | H |
| B22 | n-C₁₃H₂₇CO | 2 | 0 | H | 0 | H |
| B25 | n-C₉H₁₉CO | 0 | 0 | CONH₂ | 0 | H |
| B38 | R₁ = n-C₉H₁₁CO; | 0 | 0 | CO₂H | 0 | H |
| | R₂ = R₃ = n-C₅H₁₁CO | | | | | |
| B39 | R₁ = R₃ = n-C₅H₁₁CO; | 0 | 0 | CO₂H | 0 | H |
| | R₂ = n-C₉H₁₉CO | | | | | |

For all compounds shown: Z = Y = O; R₄ = R₅ = H; m = 0; R₈ = phosphono; R₉ = H.
*the stereochemistry of the carbon atom to which R₅ is attached is S.
**the stereochemistry of the carbon atom to which R₅ is attached is R.

In yet another embodiment, an AGP of Formula (Ia) is:

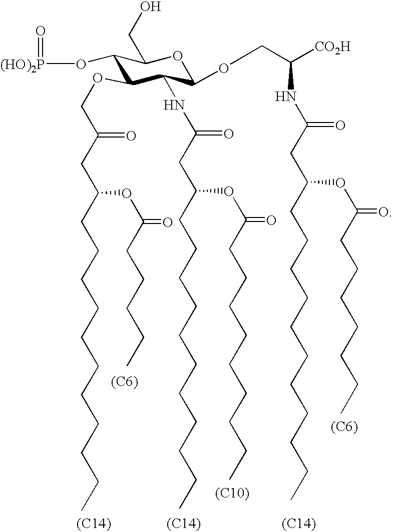

Another preferred adjuvant comprises a saponin compound, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Saponins

“Saponin,” as the term is used herein, encompasses natural and synthetic glycosidic triterpenoid compounds and pharmaceutically acceptable salts, derivatives, mimetics (e.g., isotucaresol and its derivatives) and/or biologically active fragments thereof, which possess immune adjuvant activity.

In one illustrative embodiment, saponins employed in the vaccine compositions of the present invention can be purified from Quillaja saponaria Molina bark, as described in U.S. Pat. No. 5,057,540, the disclosure of which is incorporated herein by reference in its entirety.

The adjuvant properties of saponins were first recognized in France in the 1930's. (see, Bomford et al., Vaccine 1992, 10: 572-577). Two decades later the saponin from the bark of the Quillaja saponaria Molina tree found wide application in veterinary medicine, but the variability and toxicity of these crude preparations precluded their use in human vaccines. (see, Kensil et al., In Vaccine Design: The Subunit and Adjuvant Approach; Powell, M. F., Newman, J. J., Eds.; Plenum Press: New York, 1995 pp. 525-541).

In the 1970's a partially purified saponin fraction known as Quil A was shown to give reduced local reactions and increased potency (see, Kensil et al., 1995). Further fractionation of Quil A, which consisted of at least 24 compounds by HPLC, demonstrated that the four most prevalent saponins, QS-7, QS-17, QS-18, and QS-21, were potent adjuvants (see, Kensil, C. R. Crit Rev. Ther. Drug Carrier Syst. 1996, 13, 1-55; Kensil et al., 1995). QS-21 and QS-7 were the least toxic of these. Partly because of its reduced toxicity, highly purified state (though still a mixture of no less than four compounds), (see, Soltysik, S.; Bedore, D. A.; Kensil, C. R. Ann. N.Y. Acad. Sci. 1993, 690: 392-395) and more complete structural characterization, QS-21 (3) was the first saponin selected to enter human clinical trials. (see, Kensil, 1996; Kensil et al., 1995).

QS-21 and other Quillaja saponins increase specific immune responses to both soluble T dependent and T-independent antigens, promoting an Ig subclass switch in B-cells from predominantly IgG1 or IgM to the IgG2a and IgG2b subclasses (Kensil et al., 1995). The IgG2a and IgG2b isotypes are thought to be involved in antibody dependent cellular cytotoxicity and complement fixation (Snapper and Finkelman, In Fundamental Immunology, 4th ed.; Paul, W. E., Ed.: Lippincott-Raven: Philadelphia, Pa., 1999, pp. 831-861). These antibody isotypes also correlate with a Th-1 type response and the induction of IL-2 and IFN-γ-cytokines which play a role in CTL differentiation and maturation (Constant and Bottomly, Annu. Rev. Immunology 1997, 15: 297-322). As a result, QS-21 and other Quillaja saponins are potent inducers of class I MHC-restricted CD8+ CTLs to subunit antigens (Kensil, 1996; Kensil et al., 1995).

According to an aspect of the present invention, a saponin employed in the immunostimulant composition comprises a Quillaja saponin. In one preferred embodiment of this aspect of the invention, the Quillaja saponin comprises QS-7, QS-17, QS-18 and/or QS-21.

According to another aspect of the present invention, a saponin employed in the immunostimulant composition comprises a triterpene saponin-lipophile conjugate comprising a nonacylated or desacylated triterpene saponin that includes a 3-glucuronic acid residue; and a lipophilic moiety; wherein said saponin and said lipophilic moiety are covalently attached to one another, either directly or through a linker group, and wherein said direct attachment or attachment to said linker occurs through a covalent bond between the carboxyl carbon of said 3-glucuronic acid residue, and a suitable functional group on the lipophilic residue or linker group.

The triterpene saponin can have a triterpene aglycone core structure with branched sugar chains attached to positions 3 and 28, and an aldehyde group linked or attached to position 4; and is either originally non-acylated, or require removal of an acyl or acyloyl group that is bound to a saccharide at the 28-position of the triterpene aglycone. The triterpene saponin can have a quillaic acid or gypsogenin core structure. Some saponin-lipophile conjugates useful in this invention, including GPI-0100, a quillaja saponin-lipophile conjugate, are disclosed in U.S. Pat. Nos. 5,977,081 and 6,080,725, each of which is incorporated herein by reference in its entirety. The desacylsaponin or nonacylated saponin can be selected from the group consisting of Quillaja desacylsaponin, S. jenisseensis desacylsaponin, Gypsophila saponin, Saponaria saponin, Acanthophyllum saponin and lucyoside P saponin.

The lipophilic moiety can comprise one or more residues of a fatty acid, terpenoid, aliphatic amine, aliphatic alcohol, aliphatic mercapto mono- or poly-C₂-C₄ alkyleneoxy derivative of a fatty acid, mono- or poly-C₂-C₄ alkyleneoxy derivative of a fatty alcohol, glycosyl-fatty acid, glycolipid, phospholipid or a mono-, or di-acylglycerol.

In another aspect of the present invention, the saponin employed in the immunostimulant composition comprises a saponin/antigen covalent conjugate composition.

QS-21 and other Quillaja saponins can be purified from Quillaja sponaria using standard biochemical methodologies. Briefly, aqueous extracts of Quillaja saponaria Molina bark are dialyzed against water. The dialyzed extract is lyophilized to dryness, extracted with methanol, and the methanol-soluble extract is further fractionated on silica gel chromatography and by reverse phase high pressure liquid chromatography (RP-HPLC). The individual saponins are then be separated by reverse phase HPLC. At least 22 peaks (denominated QA-1 to QA-22, also referred to herein as QS-1 to QS-21) are separable using this approach, with each peak corresponding to a carbohydrate peak and exhibiting a single band on reverse phase thin layer chromatography. The individual components can be specifically identified by their retention times on a C4 HPLC column, for example.

Preferably, the Quillaja saponins employed according to this embodiment of the invention correspond to peaks QS-7, QS-17, QS-18, and/or QS-21, as described in U.S. Pat. No. 5,057,540. In one specific embodiment of the invention, QS-21 saponin is used in accordance with this disclosure.

The substantially pure QS-7 saponin is characterized as having immune adjuvant activity and containing about 35% carbohydrate (as assayed by anthrone) per dry weight.

QS-7 has a UV absorption maxima of 205-210 nm, a retention time of approximately 9-10 minutes on RP-HPLC on a Vydac C₄ column having 5 μm particle size, 330 angstrom pore, 4.6 mm ID×25 cm L in a solvent of 40 mM acetic acid in methanol/water (58/42; v/v) at a flow rate of 1 ml/min, eluting with 52-53% methanol from a Vydac C₄ column having 5 μm particle size, 330 angstrom pore, 10 mM ID×25 cm L in a solvent of 40 mM acetic acid with gradient elution from 50 to 80% methanol, having a critical micellar concentration of approximately 0.06% in water and 0.07% in phosphate buffered saline, causing no detectable hemolysis of sheep red blood cells at concentrations of 200 μg/ml or less, and containing the monosaccharide residues terminal rhamnose, terminal xylose, terminal glucose, terminal galactose, 3-xylose, 3,4-rhamnose, 2,3-fucose, and 2,3-glucuronic acid, and apiose.

The substantially pure QS-17 saponin is characterized as having adjuvant activity and containing about 29% carbohydrate (as assayed by anthrone) per dry weight. QS-17 has a UV absorption maxima of 205-210 nm, a retention time of approximately 35 minutes on RP-HPLC on a Vydac C₄ column having 5 μm particle size, 330 angstrom pore, 4.6 mm ID×25 cm L in a solvent of 40 mM acetic acid in methanol-water (58/42; v/v) at a flow rate of 1 ml/min, eluting with 63-64% methanol from a Vydac C₄ column having 5 μm particle size, 330 angstrom pore, 10 mm ID×25 cm L in a solvent of 40 mM acetic acid with gradient elution from 50 to 80% methanol, having a critical micellar concentration of 0.06% (w/v) in water and 0.03% (w/v) in phosphate buffered saline, causing hemolysis of sheep red blood cells at 25 μg/ml or greater, and containing the monosaccharide residues terminal rhamnose, terminal xylose, 2-fucose, 3-xylose, 3,4-rhamnose, 2,3-glucuronic acid, terminal glucose, 2-arabinose, terminal galactose and apiose.

The substantially pure QS-18 saponin is characterized as having immune adjuvant activity and containing about 25-26% carbohydrate (as assayed by anthrone) per dry weight. QS-18 has a UV absorption maxima of 205-210 nm, a retention time of approximately 38 minutes on RP-HPLC on a Vydac C₄ column having 5 μm particle size, 330 angstrom pore, 4.6 mm ID×25 cm L in a solvent of 40 mM acetic acid in methanol/water (58/42; v/v) at a flow rate of 1 ml/min, eluting with 64-65% methanol from a Vydac C₄ column having 5 μm particle size, 330 angstrom pore, 10 mm ID×25 cm L in a solvent of 40 mM acetic acid with gradient elution from 50 to 80% methanol, having a critical micellar concentration of 0.04% (w/v) in water and 0.02% (w/v) in phosphate buffered saline, causing hemolysis of sheep red blood cells at concentrations of 25 μg/ml or greater, and containing the monosaccharides terminal rhamnose, terminal arabinose, terminal apiose, terminal xylose, terminal glucose, terminal galactose, 2-fucose, 3-xylose, 3,4-rhamnose, and 2,3-glucuronic acid.

The substantially pure QS-21 saponin is characterized as having immune adjuvant activity and containing about 22% carbohydrate (as assayed by anthrone) per dry weight. The QS-21 has a UV absorption maxima of 205-210 nm, a retention time of approximately 51 minutes on RP-HPLC on a Vydac C₄ column having 5 μm particle size, 330 angstrom pore, 4.6 mm ID×25 cm L in a solvent of 40 mM acetic acid in methanol/water (58/42; v/v) at a flow rate of 1 ml/min, eluting with 69 to 70% methanol from a Vydac C₄ column having 5 μm particle size, 330 angstrom pore, 10 mm ID×25 cm L in a solvent of 40 mM acetic acid with gradient elution from 50 to 80% methanol, with a critical micellar concentration of about 0.03% (w/v) in water and 0.02% (w/v) in phosphate buffered saline, causing hemolysis of sheep red blood cells at concentrations of 25 μg/ml or greater, and containing the monosaccharides terminal rhamnose, terminal arabinose, terminal apiose, terminal xylose, 4-rhamnose, terminal glucose, terminal galactose, 2-fucose, 3-xylose, 3,4-rhamnose, and 2,3-glucuronic acid.

In another embodiment of the invention, the saponin can be in the form of a saponin/antigen conjugate, as described in U.S. Pat. No. 5,583,112, the disclosure of which is incorporated herein by reference in its entirety. In this approach, one or more saponins are linked to an antigen, such that the linkage does not interfere substantially with the ability of the saponin to stimulate an immune response in the animal to which the conjugate is administered.

In another embodiment of the invention, the saponins can be modified to increase their uptake across mucous membranes, for example as described in U.S. Pat. Nos. 5,273,965, 5,443,829 and 5,650,398, the disclosures of which are incorporated herein by reference in their entireties.

In yet another embodiment, the saponins employed in the vaccine compositions of this invention comprise saponin-lipophile conjugates, as described in U.S. Pat. Nos. 5,977,081 and 6,080,725, the disclosures of which are incorporated herein by reference in its entirety. The saponin-lipophile conjugates generally comprise: (1) a non-acylated or deacylated triterpene saponin having a 3-O-glucuronic acid residue, covalently attached to: (2) a lipophilic moiety, for example, one or more fatty acids, fatty amines, aliphatic amines, aliphatic alcohols, aliphatic mercaptans, terpenes or polyethylene glycols; wherein (2) is attached to (1) via the carboxyl carbon atom present on the 3-O-glucuronic acid residue of the triterpene saponin, either directly or through an appropriate linking group.

The attachment of a lipophilic moiety to the 3-O-glucuronic acid of a saponin, such as Quillaja desacylsaponins, Silene jenisseenis, Willd's desacylsaponins, lucyoside P, and Gypsophila Saponaria and Acanthophyllum squarrosum 's saponins has been reported to enhance their adjuvant effects on humoral and cell mediated immunity. Additionally, the attachment of a lipophilic moiety to the 3-O-glucuronic acid residue of nonacylated or deacylated saponin may yield a saponin analog that is easier to purify, less toxic and/or chemically more stable, and that may possess equal or better adjuvant properties than the original saponin.

Therefore, the saponins according to this embodiment broadly comprise modified saponins, wherein said modified saponins (a) have a triterpene aglycone core structure (such as quillaic acid, gypsogenin and others) with branched sugar chains attached to positions 3 and 28, and an aldehyde group linked or attached to position 4; (b) are either originally non-acylated, or require removal of an acyl or acyloyl group that is bound to a saccharide at the 28-position of the triterpene aglycone; and (c) have a lipophilic moiety covalently attached, either directly or through a linker moiety, to the carboxylic acid of glucuronic acid at the 3-position of the triterpene aglycone. An example of such a saponin is QS-21 (3):

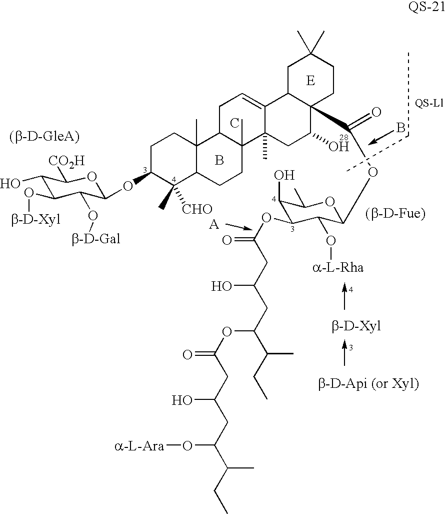

The phrases “lipophilic moiety” and “a residue of a lipophilic molecule,” as used herein, refer to a moiety that is attached by covalent interaction of a suitable functional group of one or more compounds that are non-polar or have a non-polar domain with the 3-O-glcA residue of a saponin. The lipophilic moiety can be a portion of an amphipathic compound.

An amphipathic compound is a compound whose molecules contain both polar and non-polar domains. Surfactants are examples of amphipathic compounds. Surfactants typically possess a non-polar portion that is often an alkyl, aryl or terpene structure. In addition, a surfactant possesses a polar portion that can be anionic, cationic, amphoteric or non-ionic. Examples of anionic groups are carboxylate, phosphate, sulfonate and sulfate. Examples of cationic domains are amine salts and quaternary ammonium salts. Amphoteric surfactants possess both an anionic and cationic domain. Non-ionic domains are typically derivatives of a fatty acid carboxy group and include saccharide and polyoxyethylene derivatives.

A lipophilic moiety can also comprise two or more compounds possessing non-polar domains, wherein each of the compounds has been completely bonded to a linking group, which, in turn, is covalently attached to the 3-O-glucuronic acid.

Several lipophile-containing compounds, such as aliphatic amines and alcohols, fatty acids, polyethylene glycols and terpenes, can be added to the 3-O-glcA residue of deacylsaponins and to the 3-O-glcA residue of non-acylated saponins. The lipophile may be an aliphatic or cyclic structure that can be saturated or unsaturated. By way of example, fatty acids, terpenoids, aliphatic amines, aliphatic alcohols, aliphatic mercaptans, glycosyl-fatty acids, glycolipids, phospholipids and mono- and di-acylglycerols can be covalently attached to nonacylated saponins or desacylsaponins. Attachment can be via a functional group on a lipophilic moiety that covalently reacts with either the acid moiety of the 3-glucuronic acid moiety, or an activated acid functionality at this position. Alternatively, a bifunctional linker can be employed to conjugate the lipophile to the 3-O-glcA residue of the saponin.

Illustrative fatty acids include C₆-C₂₄ fatty acids, preferably C₇-C₁₈ fatty acids. Examples of useful fatty acids include saturated fatty acids such as lauric, myristic, palmitic, stearic, arachidic, behenic, and lignoceric acids; and unsaturated fatty acids, such as palmitoleic, oleic, linoleic, linolenic and arachidonic acids.

Illustrative aliphatic amines, aliphatic alcohols and aliphatic mercaptans include amines and alcohols and mercaptans (RSH) having a straight-chained or branched, saturated or unsaturated aliphatic group having about 6 to about 24 carbon atoms, preferably 6 to 20 carbon atoms, more preferably 6 to 16 carbon atoms, and most preferably 8 to 12 carbon atoms. Examples of useful aliphatic amines include octylamine, nonylamine, decylamine, dodecylamine, hexadecylamine, sphingosine and phytosphingosine. Examples of useful aliphatic alcohols include octanol, nonanol, decanol, dodecanol, hexadecanol, chimyl alcohol and selachyl alcohol.

Illustrative terpenoids include retinol, retinal, bisabolol, citral, citronellal, citronellol and linalool.

Illustrative mono- and di-acylglycerols include mono-, and di-esterified glycerols, wherein the acyl groups include 8 to 20 carbon atoms, preferably 8 to 16 carbon atoms.

Illustrative polyethylene glycols have the formula H—(O—CH₂—CH₂)ₙ—OH, where n, the number of ethylene oxide units, is from 4 to 14. Examples of useful polyethylene glycols include PEG 200 (n=4), PEG 400 (n=8-9), and PEG 600 (n=12-14).

Illustrative polyethylene glycol fatty alcohol ethers, wherein the ethylene oxide units (n) are between 1 to 8, and the alkyl group is from C₆ to C₁₈.

A side-chain with amphipathic characteristics, i.e. asymmetric distribution of hydrophilic and hydrophobic groups, facilitates (a) the formation of micelles as well as an association with antigens, and (b) the accessibility of the triterpene aldehyde to cellular receptors. It is also possible that the presence of a negatively-charged carboxyl group in such a side-chain may contribute to the repulsion of the triterpene groups, thus allowing them a greater degree of rotational freedom. This last factor would increase the accessibility of cellular receptors to the imine-forming carbonyl group.

The desacylsaponins and non-acyl saponins may be directly linked to the lipophilic moiety or may be linked via a linking group. By the term “linking group” is intended one or more bifunctional molecules that can be used to covalently couple the desacylsaponins, non-acylated saponins or mixtures thereof to the lipophilic molecule. The linker group covalently attaches to the carboxylic acid group of the 3-O-glucuronic acid moiety on the triterpene core structure, and to a suitable functional group present on the lipophilic molecule.

Illustrative examples of linker groups which can be used to link the saponin and lipophilic molecule are alkylene diamines (NH₂—CH₂)ₙ—NH₂), where n is from 2 to 12; aminoalcohols (HO—(CH₂)ᵣ—NH₂), where r is from 2 to 12; and amino acids that are optionally carboxy-protected; ethylene and polyethylene glycols (H—(O—CH₂—CH₂)ₙ—OH, where n is 1-4) aminomercaptans and mercaptocarboxylic acids.

In yet another embodiment of the invention, the saponins employed in the compositions of the invention comprise saponin mimetics represented by the following formula (II):

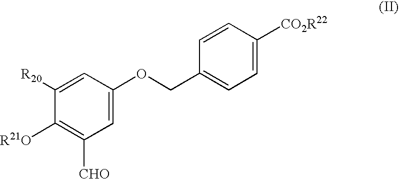

where the symbol R₂₀ represents hydrogen or —C(O)H. The symbol R²¹ represents a member selected from hydrogen, an optionally substituted C₁₋₂₀ aliphatic group, a saccharyl group, and a group represented by the formula —C(O)—[C(R²³)(R²⁴)]ₖ—COOH or —[C(R²³)(R²⁴)]ₖ—COOH, wherein each R²³ and R²⁴ independently is a member selected from hydrogen, a substituted C₁₋₁₀ aliphatic group, or an unsubstituted C₁₋₁₀ aliphatic group. The symbol k represents an integer from 1 to 5. The symbol R²² represents a member selected from hydrogen, an optionally substituted C₁₋₂₀ aliphatic group and a group represented by the formula —(CH₂)ᵣCH(OH)(CH₂)ₜOR²⁵, wherein r and t are independently 1 or 2, and R²⁵ is a C₂₋₂₀ acyl group, or a group represented by the formula

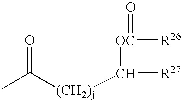

wherein j is an integer from 1 to 5, and R²⁶ and R²⁷ are independently selected from the group of hydrogen, an optionally substituted C₁₋₂₀ aliphatic group; or a pharmacologically acceptable salt thereof.

In a preferred embodiment, R²² is a substituted or unsubstituted aliphatic group having from 1 to 10 carbon atoms, more preferably from 1 to 5 carbon atoms.

In another preferred embodiment, R²² is a group represented by the formula: —(CH₂)ᵣCH(OH)(CH₂),OR₅, in which r and t are independently 1 or 2. The symbol R²⁵ is preferably an acyl group having from 2 to 10 carbon atoms, preferably from 10 to 20 carbon atoms.

In another preferred embodiment, R²⁵ is a group represented by Formula (III) wherein j is 1, 2, or 3. R²⁶ and R²⁷ are independently selected from the group of hydrogen and optionally substituted C₁₋₂₀ aliphatic groups.

Although R²⁶ and R²⁷ can be a branched-, or straight chain, saturated or unsaturated aliphatic group of substantially any length, in a preferred embodiment, R²⁶ and R²⁷ are each independently aliphatic groups having from 1 to 10 carbon atoms. In a further preferred embodiment, R²⁶ and R²⁷ are each independently aliphatic groups having from 10 to 20 carbon atoms. In a particularly preferred embodiment, at least one of R²⁶ or R²⁷ is a substituted or unsubstituted C₁₋₁₁ aliphatic group. In addition to the compounds provided above, the present invention includes pharmacologically acceptable salts of the compounds according to Formula (II).

For those embodiments of compounds of formula (II) in which R²¹ is a saccharyl group, a variety of mono-, di-, or polysaccharides are useful. In one preferred embodiment, the saccharyl group is derived from the monosaccharide glucuronic acid, and is selected from either the α- or β-forms of this saccharyl group. As shown below, the site of attachment of the saccharyl group to the remainder of the molecule can be at the reducing end (i.e., the C1 position) of the saccharyl group, as is indicated by the wavy line.

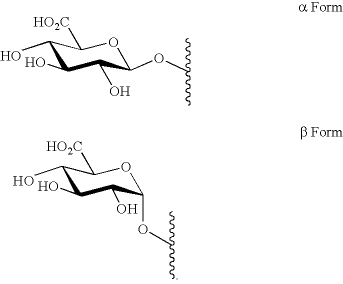

In some embodiments, it is preferred that the saccharyl group is a C₆₋₅₀ saccharyl group, more preferably a C₆₋₃₀ saccharyl group, and still more preferably a C₆₋₂₀ saccharyl group, and yet still more preferably a C₆₋₁₀ saccharyl group.

Within the above general description, a number of embodiments of compounds of formula (II) are particularly preferred. In one preferred embodiment, R²⁰, R²¹ and R²² are all hydrogens, and the compound is isotucaresol, represented by Formula (IV):

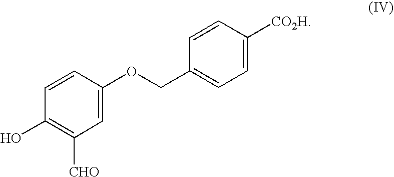

In another preferred embodiment, R²⁰ is hydrogen, R²¹ is a β-D-glucuronic acid group, R²² is hydrogen, and the compound is represented by Formula (V):

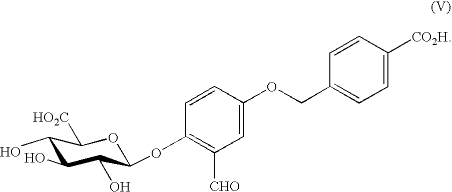

In one embodiment, R²⁰ is hydrogen, R² is a succinoyl group (i.e., R²¹=—C(O)—[C(R²³)(R²⁴)]ₖ—COOH, wherein R²³ and R²⁴ are hydrogen; k is 2 and R²² is hydrogen. The compound is represented by Formula (VI):

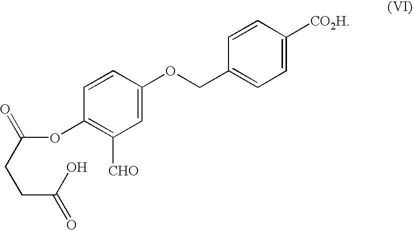

In one embodiment of formula (VI) compounds, R²⁰ is hydrogen, R²¹ is a β-D-glucuronic acid group, and R²² is an 1-O-acyl-sn-glyceryl group (sn=stereospecifically numbered; see, Carb. Res. 1998, 312, 167), and the compound is represented by Formula (VII):

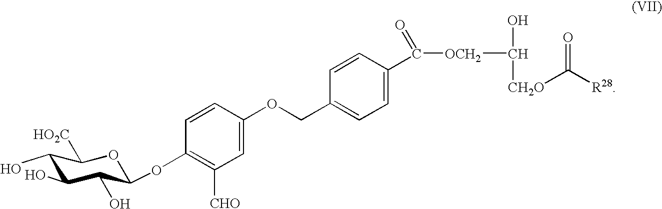

In one embodiment, the acyl group of the 1-O-acyl-sn-glyceryl moiety is acetyl (e.g., R²⁸ in Formula VII is methyl; compound 6a), and in another embodiment, octanoyl (R²⁸ is heptyl; compound 6b), and in one embodiment, tetradecanoyl (R²⁸ is tridecyl; compound 6c).

The amphipathic aldehydes (IV)-(VI) as saponin mimetics are based on isotucaresol (IV) as an open-chain analog of quillaic acid (1) which is substituted with lipophilic and/or hydrophilic domains. The design of isotucaresol as a pharmacophore of 1 is based on the premise that saponins are more structurally complex than is necessary for optimal adjuvant effects. Like steroids, the ABC-ring junctures of quillaic acid are all-trans, making the molecule relatively rigid and flat, and thus amenable to molecular mimicry by aromatic seco derivatives. Isotucaresol is an aromatic “triseco” derivative of quillaic acid in which elements of three rings (B, C, E) of the triterpene have been removed but the spatial relationship of key functionality has been maintained.

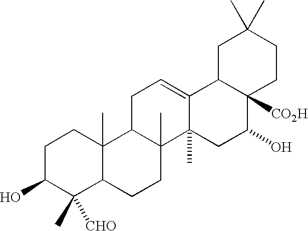

### Quillaic Acid (1)

The significance of having two reactive aldehyde moieties on the A-ring of isotucaresol provides the potential for simultaneous engagement of both formyl groups in imine formation with the multiple lysyl E-amino groups (see, Wyss et al., Science 1995, 269: 1273-1278) clustered in the CD2 cell-surface glycoprotein present on T lymphocytes. CD2 is believed to be the principle receptor for Schiff base-mediated costimulation of T-cells (Rhodes, 1996). Multivalent ligand-receptor interactions are common in biological systems and, in the context of T-cell activation, may help to explain not only the immunogenicity of MAA-adducted peptides but also the success of a recent cancer vaccine strategy (see, Apostolopoulos et al., Proc. Natl. Acad. Sci., U.S.A. 1995, 92: 10128-10132) employing formylated mucins.

In another aspect, the present invention includes a compound represented by the Formula II(a):

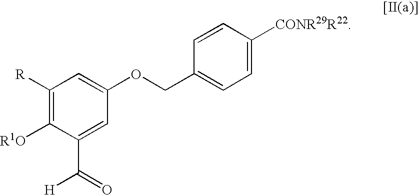

R²² and R²⁹ are independently selected and the symbol R²⁹ represents a member as described above for R²². Compounds of Formula 11(a) are useful as adjuvants and immunoeffectors as described herein for compounds of Formulas (Ia)-(Ic).

In another aspect, the present invention provides a compound represented by the Formula II(b):

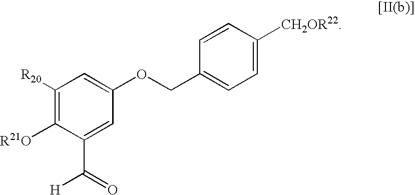

Compounds of Formula II(b) are useful as adjuvants and immunoeffectors as described herein for compounds of Formula (Ia)-(Ic).

By covalently bonding an antigen to an extrinsic adjuvant (immunomodulator) such as a compound of Formula (II, Ia or IIb), a discrete molecule is produced which exhibits a surprisingly unexpected enhanced adjuvanting effect on the antigen which is greater than the adjuvanting effect attainable in the absence of such covalent bonding, as in a mixture of the two components (i.e., the antigen and a compound of Formula (II, IIa or IIb). A further enhanced adjuvanting effect may be attained for such covalently-bonded antigen by incorporating a mineral salt adjuvant with such compounds. The mineral salt adjuvant preferably comprises aluminum hydroxide or aluminum phosphate, although other known mineral salt adjuvants, such as calcium phosphate, zinc hydroxide or calcium hydroxide, may be used.

Aqueous solubility is a desirable characteristic of adjuvant-active saponins and aids in vaccine formulation and efficacy (Kensil, 1996). Unlike oil-based emulsions and mineral salt adjuvants which can denature antigens and prevent protective effects, saponins are non-denaturing adjuvants due to their high aqueous solubility. Their high water solubility also obviates extensive homogenation procedures required for emulsion-type adjuvants, permitting simple mixing of aqueous adjuvant and antigen solutions prior to immunization. Although saponins exhibit a great deal of structural variability in the glycosides attached to C-3 and C-28 of the quillaic acid aglycon unit, the minimal carbohydrate requirement for adjuvanticity (and aqueous solubility) either alone or in formulation (with ISCOMs, alum, etc.) appears to be a glycosidically linked D-glucuronic acid (β-D-GlcA) moiety at C-3 (see, Bomford et al., Vaccine 1992, 10: 572-577; So et al., 1997). Thus, a D-glucuronic acid moiety, glycosidically linked to the phenol group of isotucaresol-itself sparingly soluble at physiologic pH-enhances both aqueous solubility and adjuvanticity, partly by virtue of a second ionizable carboxyl group. Water-soluble O-glycosides of simple hydroxybenzaldehydes (e.g., helicin (31)) not only occur in nature but readily form stable Schiff-base derivatives as well (see, The Merck Index, 12th ed.; Merck & Co., Inc.: Whitehouse Station, N.J., 1996) The synthetically simpler succinate (VI) is also useful since succinic acid constitutes a simple 4-carbon isostere for the glucuronic acid moiety and has been used to impart triterpenes with aqueous solubility (see, Gottfried and Baxendale, U.S. Pat. No. 3,070,623, 1962).

It is important to note that chemical modification of the glucuronic carboxyl of QS-21 does not significantly alter adjuvant activity (Soltysik et al., 1995). Thus, the carboxyl group offers a unique site for attachment of a lipophilic fatty acid domain or a poorly immunogenic peptide. In fact, the attachment of simple lipophilic moieties to the glucuronic acid of deacylated Quillaja saponin or saponins lacking fatty acid domains was recently shown to enhance humoral and cell-mediated immunity (see, Marciani, WO 98/52573, 1998; and U.S. Pat. No. 6,080,725). A peptide determinant linked to the glucuronic carboxyl of a compound of Formula V (or the more lipophilic derivatives according to compounds 6a-6c) would also confer favorable solubility characteristics and potentially provide synthetic vaccines with built-in adjuvanticity. Increased immunogenicity has been observed for lipophilic Quillaja saponins covalently linked to peptide antigens via the glucuronic carboxyl (see, Kensil et al., In Vaccines 92; Brown, F., Chanock, R. M., Ginsberg, H. S., Lemer, R. A., Eds.; Cold Spring Harbor Laboratory Press: Plainview, N.Y., 1992; pp. 35-40).

While not wishing to be bound by ththeheory or rationale for using hydrophilic Schiff-base-forming compounds lacking fatty acyl groups (i.e., compounds according to Formulae V and VI as adjuvants and immunoeffectors, the use of these compounds deserves further comment. In the case of QS-21 the fatty acid domain, common also to QS-17 and QS-18, plays a critical role: controlled alkaline hydrolysis to give either a desacyl saponin (cleavage at site A in 3) or a quillaic acid derivative (cleavage at the site B) shows that neither of these two hydrolysis products nor the intact fatty acid domain enhance antibody titers or antigen-specific CTLs to ovalbumin when formulated in phosphate buffered saline (PBS) (see, Kensil et al., 1996; Kensil et al., 1992). This and other evidence suggests that antigen binding through hydrophobic interactions is reduced or eliminated when the fatty acid domain is absent. However, a recent study with the QS-21 “B fragment” isolated from unmodified crude Quillaja extract showed that this saponin (designated QS-L1, see, QS-21 partial structure) boosted humoral and cellular immune responses to recombinant hepatitis B surface antigen (rHBsAg) when administered in the presence of alum precipitated antigen. In fact, QS-L1 induced a greater total IgG response in mice than QS-21 to alum-precipitated HBsAg (So et al., 1997). These results suggest the importance of charge interaction between alum, anionic adjuvants, and peptide antigens.

The importance of the fatty acid domain to saponin adjuvanticity is further obscured by the recent structure elucidation of the hydrophilic saponin QS-7 (Kensil et al., 1998). QS-7 is a bisdesmosidic saponin possessing branched sugar units at C-3 and C-28 of quillaic acid similar to those of QS-21, but in contrast possesses an acetyl group in lieu of a large lipid domain on the fucose ring. Like QS-21, QS-7 is a potent inducer of cell-mediated and humoral responses to a variety of antigens, but lacks the characteristic hemolytic activity of saponins towards red blood cells (Kensil, 1996; Kensil et al., 1998). Hemolytic activity-thought to be due to the ability of saponin to intercalate into cell membranes and form a hexagonal array of pores involving cholesterol-complexed saponin molecules-does not correlate with adjuvant activity, however: QS-7 is non-hemolytic whereas digitonin, an adjuvant-inactive steroidal saponin, is highly hemolytic (Kensil, 1996; Kensil et al., 1998; see, Kensil et al., J. Immunol. 1991, 146: 431-437). Thus, CTL induction by exogenous soluble antigen does not appear to be closely associated with either saponin-induced pore formation or the presence of a complex lipophilic domain.

In addition to contributing to the greater toxicity of QS-21 and other lipophilic saponins, the complex fatty acid domain comprising two 3,5-dihydroxy-6-methyl-octanoic acid (DHMO) residues imparts considerable instability to lipophilic saponins. For example, a rapid reversible migration of the DHMO domain occurs between the 3- and 4-hydroxyl groups of fucose in QS-21, confounding purification and purity analysis as well as structure/function assessment (see, Cleland et al., J. Pharm. Sci. 1996, 85: 22-28).

This intramolecular transesterification can be ascribed to the known lability of hydroxy esters (see, Sadekov et al., Russ. Chem. Rev. (Eng. Transl.) 1970, 39: 179-195) (to nucleophilic attack by a vicinal hydroxyl in 3, for example). For the same reason, base-catalyzed deacylation is a significant degradation process for QS-21 in aqueous solution, thus limiting the formulations and storage conditions with which QS-21 can be used (Kensil et al., 1995; Cleland, 1996).

Accordingly, the lipophilic derivatives (compounds 6a-c) wherein an sn-glycerol unit (same C-2 relative stereochemistry as D-fucose) has been selected as an open-chain analog of the fucose ring and simple fatty acid residues as stable substitutes for the complex DHMO residues of QS-21; acetate (compound 6a) is an analog of the more hydrophilic and less toxic QS-7. The structural relationship between compounds according to compound 6a and QS-21 is shown in bold in 3.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol^{R} to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 and is disclosed in WO 00/09159. Preferably the formulation additionally comprises an oil in water emulsion and tocopherol.

Additional illustrative adjuvants for use in the pharmaceutical compositions of the invention include Montamide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula

HO(CH₂CH₂O)ₙ-A-R, (I)

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG.

Definitions

In the above formulas:

The term “acyl” refers to those groups derived from an aliphatic organic acid by removal of the hydroxy portion of the acid. Accordingly, acyl is meant to include, for example, acetyl, propionyl, butyryl, decanoyl, pivaloyl, and the like. A “C₁-C₂₀ acyl group” thus is an acyl group having from 1 to 20 carbons.

The term “aliphatic,” means, unless otherwise stated, a non-aromatic straight or branched chain, or cyclic, hydrocarbon moiety, saturated or mono- or poly-unsaturated, including such a moiety that contains both cyclical and chain elements, having the designated number of carbon atoms (i.e. C₁-C₁₀ means having from one to ten carbons). Types of saturated hydrocarbon radicals include alkyl, alkylene, cycloalkyl or cycloalkyl-alkyl groups such as methyl, ethyl, n-propyl, isopropyl, n-butyl, t-butyl, isobutyl, sec-butyl, methylene, ethylene, n-butylene, cyclopropyl, and cyclopropylmethyl.

An unsaturated aliphatic group is one having one or more double and/or triple bonds. Examples of unsaturated aliphatic groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, cyclohexenyl, and cyclohexadienyl.

A “C₁-C₂₀ aliphatic group” is a substituted or unsubstituted aliphatic group having from 1 to 20 carbons. Similarly, a “Cl ₁ aliphatic group” is a substituted or unsubstituted aliphatic group having 11 carbons.

The term “oxyaliphatic” refers to those aliphatic groups attached to the remainder of the molecule via an oxygen atom.

The terms “halo” or “halogen,” by themselves or as part of another substituent, mean, unless otherwise stated, a fluorine, chlorine, bromine, or iodine atom. In compounds having halogen substituents, the halogens may be the same or different.

Substituents for the aliphatic groups can be a variety of groups selected from: —OR′, ═O, ═S, ═NR′, ═N—OR′, —NR′R″, —SR′, -halogen, —SiR′R″R′″, —OC(O)R′, —C(O)R′, —CO2R′, —CONR′R″, —OC(O)NR′R″, —NR″C(O)R′, —NR′—C(O)NR″R′″, —NR″C(O)2R′, —NR—C(NRR′R″)═NR′″, —NR′C(NR′R″)═NR′″, —NR—C(NR′R″)═NR′″, —S(O)R′, —S(O)2R′, —S(O)2NR′R″, —NRSO2R′, —CN and —NO₂ in a number ranging from zero to (2m′+1), where m′ is the total number of carbon atoms in such radical and R′, R″ and R′″ each independently refer to hydrogen or (C₁-C₄) aliphatic groups. When a compound of the invention includes more than one R group, for example, each of the R groups is independently selected, as are each R′, R″ and R′″ groups when more than one of these groups is present. When R′ and R″ are attached to the same nitrogen atom, they can be combined with the nitrogen atom and optionally an additional heteroatom to form a 5-, 6-, or 7-membered ring. For example, —NR′R″ is meant to include 1-pyrrolidinyl and 4-morpholinyl. From the above discussion of substituents, one of skill in the art will understand that the term “aliphatic” is meant to include groups such as haloaliphatic (e.g., —CF₃, CClF₂, and —CH₂CF₃).

The term “saccharyl” refers to those groups derived from a sugar, a carbohydrate, a saccharide, a disaccharide, an oligosaccharide, or a polysaccharide molecule by removal of a hydrogen or a hydroxyl group. Accordingly, saccharyl groups (e.g., glucosyl, mannosyl, etc.) can be derived from molecules that include, but are not limited to, glucuronic acid, lactose, sucrose, maltose, allose, alltrose, glucose, mannose, idose, galactose, talose, ribose, arabinose, xylose, lyxose, threose, erythrose, β-D-N-Acetylgalactosamine, β-D-N-Acetylglucosamine, fucose, sialic acid, etc. A “C₆-C₂₀ saccharyl group” is a substituted (e.g. acylated saccharyl, alkylated saccharyl, arylated saccharyl, etc.) or unsubstituted saccharyl group having from 6 to 20 carbons. An example of a saccharyl group is a radical formed by the removal of the hydroxyl on the C1 position of glucuronic acid as represented by the formula:

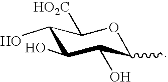

The wavy bond indicates where the glucuronide radical (i.e., a glucuronic acid group) would be attached to another substituent, e.g., an aglycon unit. Thus, saccharyl groups include sugar molecules where the hydroxyl on the C1 position has been removed.

The term “pharmaceutically acceptable salts” is meant to include salts of the compounds in question that are prepared with relatively nontoxic acids or bases, depending on the particular substituents found on the compounds described herein. When compounds of the present invention contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When compounds of the present invention contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, compounds which are in a prodrug form of the saponins or aminoalkyl glucosaminide phosphates may be included in the compositions of this invention. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds usable in compositions of the present invention can exist in unsolvated forms as well as solvated forms, including hydrated forms. In general, the solvated forms are equivalent to unsolvated forms and are encompassed within the scope of the present invention. Certain compounds usable in compositions of the present invention may exist in multiple crystalline or amorphous forms. In general, all physical forms are equivalent for the uses contemplated by the present invention and are intended to be within the scope of the present invention.

Certain compounds usable in compositions of the present invention possess asymmetric carbon atoms (optical centers) or double bonds; the racemates, diastereomers, geometric isomers and individual isomers are encompassed within the scope of the present invention.

The chemical compounds in compositions of the present invention may exist in (+) and (−) forms as well as in racemic forms. Racemic forms can be resolved into the optical antipodes by known methods and techniques. One way of separating the racemic forms is exemplified by the separation of racemic amines by conversion of the racemates to diastereomeric salts of an optically active acid. The diastereomeric salts are resolved using one or more art recognized methods. The optically active amine is subsequently liberated by treating the resolved salt with a base. Another method for resolving racemates into the optical antipodes is based upon chromatography on an optical active matrix. Racemic compounds used in compositions of the present invention can thus be resolved into their optical antipodes, e.g., by fractional crystallization of d- or l-tartrates, -mandelates, or -camphorsulfonate) salts for example.

Such compounds may also be resolved by the formation of diastereomeric amides by reaction with an optically active carboxylic acid such as that derived from (+) or (−) phenylalanine, (+) or (−) phenylglycine, (+) or (−) camphanic acid or the like. Alternatively, they may be resolved by the formation of diastereomeric carbamates by reaction of the chemical compound with an optically active chloroformate or the like.

Additional methods for the resolving the optical isomers are known in the art. Such methods include those described by Collet and Wilen, ENANTIOMERS, RACEMATES, AND RESOLUTIONS, John Wiley and Sons, New York (1981).

Moreover, some of the compounds usable in compositions of the invention can exist in syn- and anti-forms (Z- and E-form), depending on the arrangement of the substituents around a double bond. A chemical compound in a composition of the present invention may thus be the syn- or the anti-form (Z- and E-form), or it may be a mixture thereof.

The compounds usable in these compositions may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of such compounds, whether radioactive or not, are intended to be encompassed within the scope of the present invention.

Polynucleotides

As noted above, recombinant viruses employed in the compositions and methods of the present invention may comprise one or more polynucleotide sequences that express one or more polypeptides. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotides of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

Polynucleotides may comprise a native sequence (i.e., an endogenous sequence that encodes a polypeptide/protein of the invention or a portion thereof) or may comprise a sequence that encodes a variant or derivative, preferably and immunogenic variant or derivative, of such a sequence.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompass homologous genes of xenogenic origin.

In certain preferred embodiments, the polynucleotides described above, e.g., polynucleotide variants, fragments and hybridizing sequences, encode polypeptides that are immunologically cross-reactive with a polypeptides described herein above. In other preferred embodiments, such polynucleotides encode polypeptides that have a level of immunogenic activity of at least about 50%, preferably at least about 70%, and more preferably at least about 90% of that for a polypeptides described herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

Polypeptides

Recombinant viruses employed in the compositions and methods of the present invention may express one or more recombinant polypeptides having immunogenic and/or therapeutic activity in the treatment of cancer and/or infectious disease.

As used herein, the term “polypeptide” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof Particular polypeptides of interest in the context of this invention are amino acid subsequences comprising epitopes, i.e., antigenic determinants substantially responsible for the immunogenic properties of a polypeptide and being capable of evoking an immune response.

As detailed in the Examples presented herein below, one exemplary polypeptide suitable for expression from a recombinant virus is the tuberculosis antigen TbH9. One of skill in the art will recognize, however, that many other polypeptides may be suitably employed in the compositions and methods of the present invention.

The polypeptides of the present invention are sometimes herein referred to as tumor proteins or tumor polypeptides, as an indication that their identification has been based at least in part upon their increased levels of expression in tumor samples. Thus, a “tumor polypeptide” or “tumor protein,” refers generally to a polypeptide sequence of the present invention, or a polynucleotide sequence encoding such a polypeptide, that is expressed in a substantial proportion of tumor samples, for example preferably greater than about 20%, more preferably greater than about 30%, and most preferably greater than about 50% or more of tumor samples tested, at a level that is at least two fold, and preferably at least five fold, greater than the level of expression in normal tissues, as determined using a representative assay provided herein. A tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with a cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

In one preferred embodiment, the polypeptide fragments and variants provided by the present invention are immunologically reactive with an antibody and/or T-cell that reacts with a full-length polypeptide specifically set forth herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | B | GAA GAG |
| Phenylalanine | Phe | F | UUC UUU |
| Glycine | Gly | C | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUG CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (˜3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Saitou, N. Nei, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a xenogeneic polypeptide that comprises an polypeptide having substantial sequence identity, as described above, to the human polypeptide (also termed autologous antigen) which served as a reference polypeptide, but which xenogeneic polypeptide is derived from a different, non-human species. One skilled in the art will recognize that “self” antigens are often poor stimulators of CD8+ and CD4+ T-lymphocyte responses, and therefore efficient immunotherapeutic strategies directed against tumor polypeptides require the development of methods to overcome immune tolerance to particular self tumor polypeptides. For example, humans immunized with prostase protein from a xenogeneic (non human) origin are capable of mounting an immune response against the counterpart human protein, e.g. the human prostase tumor protein present on human tumor cells.

More particularly, the invention is directed to mouse, rat, monkey, porcine and other non-human polypeptides which can be used as xenogeneic forms of human polypeptides set forth herein, to induce immune responses directed against tumor polypeptides of the invention.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4+ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Pharmaceutical Compositions

In additional embodiments, the present invention concerns formulation of one or more of the recombinant viruses and immunostimulant compositions that further comprise one or more pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the compositions described herein in combination with a physiologically acceptable carrier. In certain preferred embodiments, the pharmaceutical compositions of the invention may be employed for use in prophylactic and therapeutic vaccine applications. Vaccine preparation is generally described in, for example, M. F. Powell and M. J. Newman, eds., “Vaccine Design (the subunit and adjuvant approach),” Plenum Press (NY, 1995).

Illustrative immunogenic compositions, e.g., vaccine compositions, of the present invention comprise a recombinant virus including a polynucleotide encoding one or more polypeptides, as described above, such that the polypeptide is generated in vivo. As noted above, the compositions may be administered within any of a variety of delivery systems known to those of ordinary skill in the art. Indeed, numerous viral-based gene delivery techniques are well known in the art, such as those described by Rolland, Crit. Rev. Therap. Drug Carrier Systems 15:143-198, 1998, and references cited therein. Appropriate viral expression systems will, of course, contain the necessary regulatory DNA regulatory sequences for expression in a patient (such as a suitable promoter and terminating signal).

In another embodiment of the invention, recombinant viruses may be administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, compositions of the present invention may be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as viral particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

In another illustrative embodiment, calcium phosphate core particles are employed as carriers, vaccine adjuvants, or as controlled release matrices for the compositions of this invention. Exemplary calcium phosphate particles are disclosed, for example, in published patent application No. WO/0046147.

The pharmaceutical compositions of the invention will often further comprise one or more buffers (e.g., neutral buffered saline or phosphate buffered saline), a carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature Mar. 27, 1997;386(6623):410-4; Hwang et al., Crit Rev Ther Drug Carrier Syst 1998;15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. Salts of the compounds of this invention may be prepared and used in formulations in a lyophilized form for convenience.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release Mar. 2, 1998;52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroethylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol July 1998; 16(7):307-21; Takakura, Nippon Rinsho March 1998; 56(3):691-5; Chandran et al., Indian J Exp Biol. August 1997; 35(8):801-9; Margalit, Crit Rev Ther Drug Carrier Syst. 1995;12(2-3):233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J. Biol. Chem. Sep. 25, 1990;265(27):16337-42; Muller et al., DNA Cell Biol. April 1990; 9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, the use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. December 1998; 24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988;5(1):1-20; zur Muhlen et al., Eur J Pharm Biopharm. March 1998;45(2):149-55; Zambaux et al. J Controlled Release. Jan. 2, 1998;50(1-3):31-40; and U.S. Pat. No. 5,145,684.

Cancer Therapeutic Methods

Immunologic approaches to cancer therapy are based on the recognition that cancer cells can often evade the body's defenses against aberrant or foreign cells and molecules, and that these defenses might be therapeutically stimulated to regain the lost ground, e.g. pgs. 623-648 in Klein, Immunology (Wiley-Interscience, New York, 1982). Numerous recent observations that various immune effectors can directly or indirectly inhibit growth of tumors has led to renewed interest in this approach to cancer therapy, e.g. Jager, et al., Oncology 2001;60(1):1-7; Renner, et al., Ann Hematol December 2000;79(12):651-9.

Four-basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins that are responsible for lysing and processing the immunoglobulin-coated target invader cells; iii) natural killer lymphocytes having two mechanisms for the destruction of tumor cells, antibody-dependent cellular cytotoxicity and natural killing; and iv) T-lymphocytes possessing antigen-specific receptors and having the capacity to recognize a tumor cell carrying complementary marker molecules (Schreiber, H., 1989, in Fundamental Immunology (ed). W. E. Paul, pp. 923-955).

Cancer immunotherapy generally focuses on inducing humoral immune responses, cellular immune responses, or both. Moreover, it is well established that induction of CD4⁺ T helper cells is necessary in order to secondarily induce either antibodies or cytotoxic CD8⁺ T cells. Polypeptide antigens that are selective or ideally specific for cancer cells, particularly cancer cells, offer a powerful approach for inducing immune responses against cancer, and are an important aspect of the present invention.

Therefore, in further aspects of the present invention, the pharmaceutical compositions described herein may be used to stimulate an immune response against cancer. Within such methods, the pharmaceutical compositions described herein are administered to a patient, typically a warm-blooded animal, preferably a human. A patient may or may not be afflicted with cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

The following Example is offered by way of illustration and not by way of limitation.

### EXAMPLE

### Compositions Comprising MPL in Conjunction with a Recombinant Adenovirus

The example illustrates the use of monophosphoryl lipid A (MPL) in an aqueous formulation with a recombinant adenovirus to augment the immune responses to the Mycobacterium tuberculosis antigen, TbH9 (Mtb39A).

TB antigen TbH9 was subcloned in to a recombinant E1 and E3 deleted, replication-defective adenovirus, serotype 5, vector (Ad5-TbH9). 10E5 or 10E6 plaque forming units (pfu) of Ad5-TbH9 was admixed with 10 μg of an aqueous formula of MPL (MPL-AF). This admixture was injected either subcutaneously or intradermally into C57BL/6 mice to assess antigen specific immune responses. Spleens and sera were harvested 7 weeks later and used to assay for serum antibodies and for cytotoxic T-lymphocyte (CTL) and interferon-gamma (IFN-γ) responses in splenocytes.

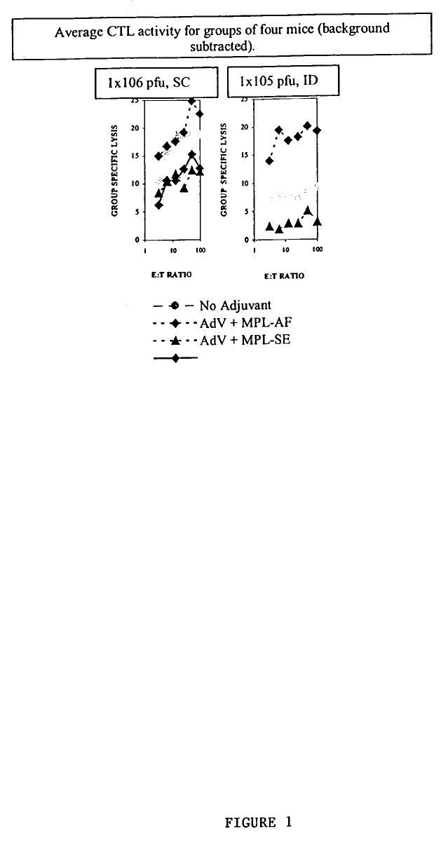

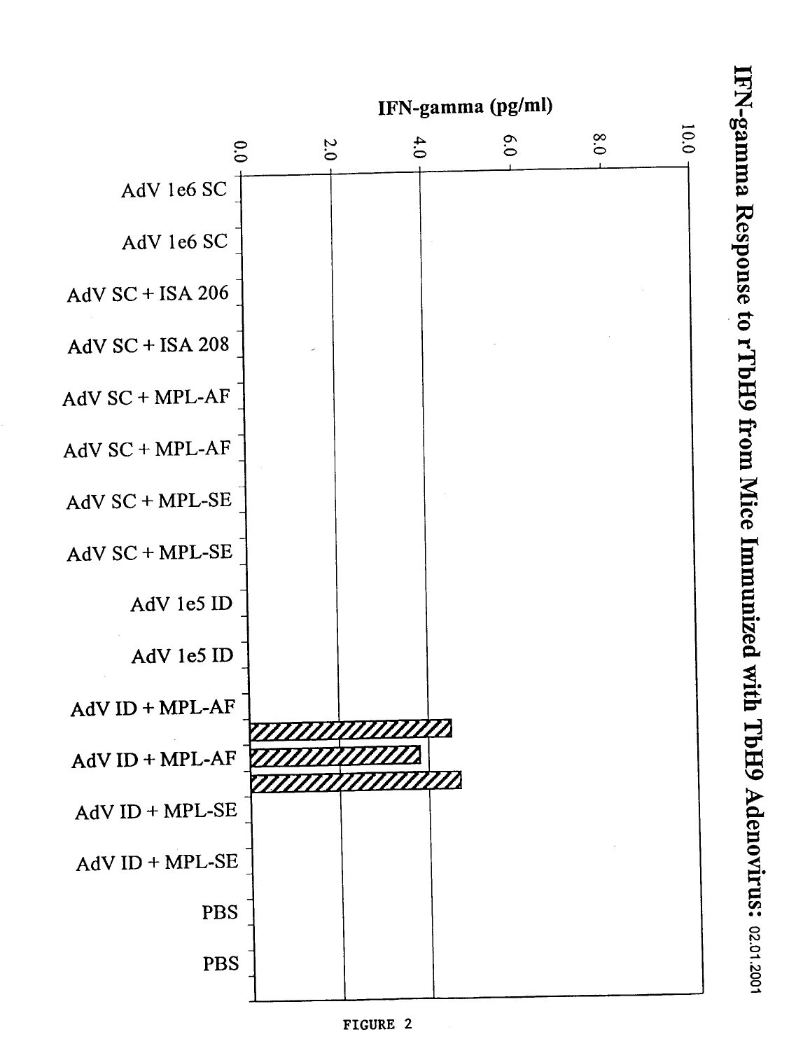

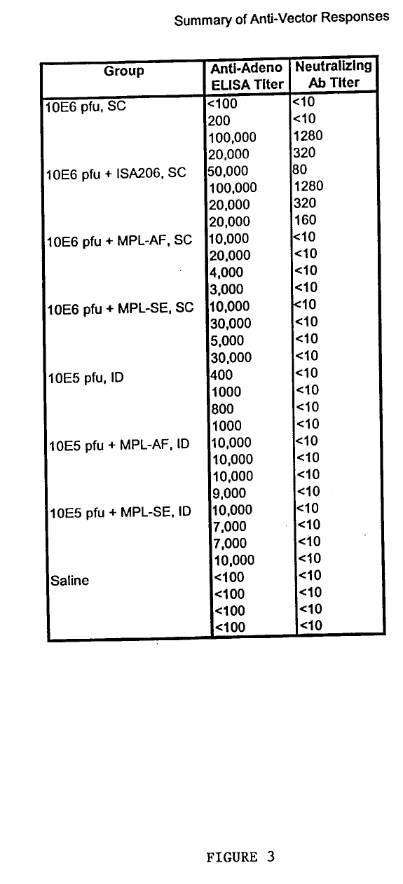

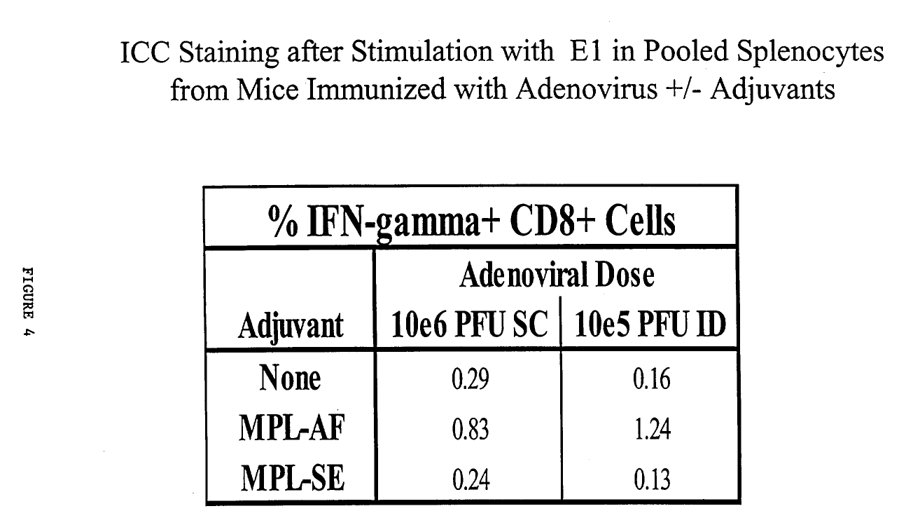

The results of those assays are found in FIGS. 1-4. MPL-AF enhanced TbH9-specific CTL and IFN-γ responses (FIGS. 1 and 2). ELISA analysis showed that antibody titers to the adenovirus were also increased in the presence of adjuvant, but this did not result in increased viral neutralizing antibody titer (FIG. 3). Intracellular cytokine (ICC) staining assays analyzed by fluorescence activated cell sorting (FACS) confirmed that MPL-AF increased IFN-γ production in CD8-positive T-cells (FIG. 4).

These data show that a low dose of adenovirus by itself does not induce a significant immune response in vivo, but when combined with MPL-AF, the CTL and IFN-γ response is enhanced. The combination of the adenovirus vector with MPL: (1) increases the efficacy of the adenovirus vaccine, (2) permits the use of lower adenovirus titers to achieve efficacious immune responses, (3) permits the use of lower adenovirus titers in priming immunizations thereby substantially reducing and/or eliminating the neutralizing response to the adenorecombinant virus and, consequently, allowing for subsequent boosting immunizations with the same adenorecombinant virus.

In a second series of experiments, 1e6 or 1e7 pfu of Ad5-TbH9 was admixed with 10 μg MPL-AF and was injected intradermally into four groups of C57BL/6 mice. Spleens and sera were harvested five weeks later and assayed for TbH9-specific CD4 cells (interferon γ ELISA after in vitro re-stimulation with recombinant TbH9 protein) and CD8 cells (FACS analysis of intracellular staining for INF-γ after overnight stimulation with EL4 cells expressing TbH9). Sera were assayed for binding antibodies to adenovirus by ELISA and for neutralizing activity by an in vitro infectivity assay.

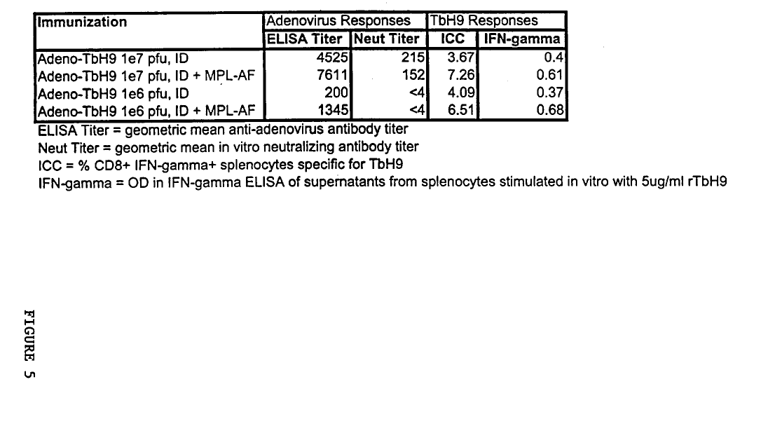

MPL-AF admixed with recombinant adenovirus enhanced the CD4+ and CD8+ immune responses to the antigen TbH9, while not enhancing antibody titers that neutralize infection of the adenovirus vector, FIG. 5.

In a third series of experiments, I e6 of Ad5-TbH9 was admixed with 10 μg of an AGP in 2% TEoA and was injected intradermally into four groups of C57BL/6 mice. The AGPs used were compounds B15, B19, B20, B38 and B39 described above. These are compounds of Formula (Ia). Spleens and sera were harvested four weeks later and assayed for TbH9-specific CD4 cells (interferon γ ELISA after in vitro re-stimulation with recombinant TbH9 protein) and CD8 cells (ELISPOT analysis of intracellular staining for INF-γ after overnight stimulation with EL4 cells expressing TbH9). Sera were assayed for binding antibodies to adenovirus by ELISA and for neutralizing activity by an in vitro infectivity assay.

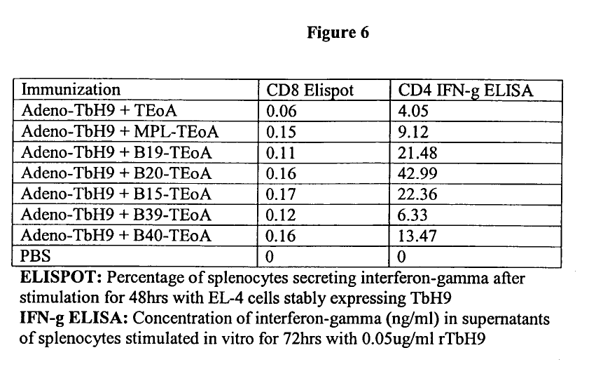

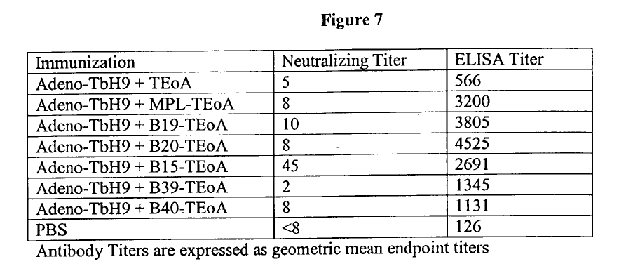

Like MPL, AGPs admixed with recombinant adenovirus enhanced the CD4+ and CD8+ immune responses to the antigen TbH9, while not enhancing antibody titers that neutralize infection of the adenovirus vector, FIGS. 6 and 7.

Such an admixture allows for a lower dose of adenovirus to be delivered in the primary inoculation while still maintaining a robust immune response. This has two potential benefits: (1) a lower primary dose allows for increased efficacy of a secondary adenovirus immunization, due to low or undetectable levels of adenovirus neutralizing antibodies, and (2) increased safety due to inoculation of lower numbers of adenovirus particles.

Immunizations with recombinant adenovirus is a potent means of eliciting a high frequency CD8+ T-cell response together with robust CD4+ T-cell and humoral responses. The use of adenovirus vaccines is limited, however, by neutralizing antibody responses to the vector which restricts the ability to boost with the same construct. In addition there are safety concerns associated with delivery of high doses of adenovirus particles to humans. Using an adjuvant, such as MPL or AGPs, in an adenovirus vaccine enhances the immune response and lowers the effective dose of the adenovirus vaccine.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A composition comprising a recombinant virus and an immunostimulant wherein said recombinant virus encodes a non-viral polypeptide and wherein said composition elicits an enhanced immune response to said polypeptide as compared to an immune response resulting from immunization with said recombinant virus in the absence of said immunostimulant in a mammal immunized with said composition.

2. A composition according to claim 1 wherein the composition elicits an enhanced immune response to said polypeptide without enhancing the neutralizing antibody response to said recombinant virus as compared to the neutralizing antibody response resulting from immunization with said recombinant virus in the absence of said immunostimulant in a mammal immunized with said composition

2. The composition of claim 1 wherein said recombinant virus is selected from the group consisting of an adenovirus, an adeno-associated virus (AAV), a pox virus, and an alphavirus.

3. The composition of claim 2 wherein said pox virus is selected from the group consisting of a vaccinia virus and an avian poxvirus.

4. The composition of claim 2 wherein said recombinant virus is an adenovirus.

5. The composition of claim 1 wherein said immunostimulant comprises one or more adjuvants.

6. The composition of claim 5 wherein said adjuvant is selected from the group consisting of Freund's Incomplete Adjuvant; Freund's Complete Adjuvant; Merck Adjuvant 65; AS-2; aluminum hydroxide gel; aluminum phosphate; a salt of calcium, iron or zinc; an insoluble suspension of acylated tyrosine acylated sugars; cationically or anionically derivatized polysaccharides; polyphosphazenes; biodegradable microspheres; aminoalkyl glucosaminide phosphates, monophosphoryl lipid A, saponins, water/oil adjuvants, and mixtures thereof.

7. The composition of claim 6 wherein said adjuvant comprises monophosphoryl lipid A.

8. The composition of claim 6 wherein said adjuvant comprises an aminoalkyl glucosaminide phosphate.

9. The composition of claim 8 wherein the aminoalkyl glucosaminide phosphate has the formula 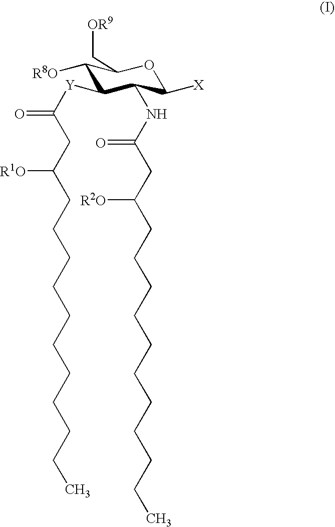
and pharmaceutically acceptable salts and derivatives thereof, wherein Y is —O— or —NH—; R¹ and R² are each independently selected from saturated and unsaturated (C₂-C₂₄) aliphatic acyl groups; R⁸ is —H or —PO₃R¹¹R¹², wherein R¹¹ and R¹² are each independently —H or (C₁-C₄) aliphatic groups; R⁹ is —H, —CH₃ or —PO₃R¹³R¹⁴, wherein R¹³ and R¹⁴ are each independently selected from —H and (C₁-C₄) aliphatic groups; and wherein at least one of R⁸ and R⁹ is a phosphorus-containing group, but R⁸ and R⁹ are not both phosphorus-containing groups; and X is a group selected from the formulae: 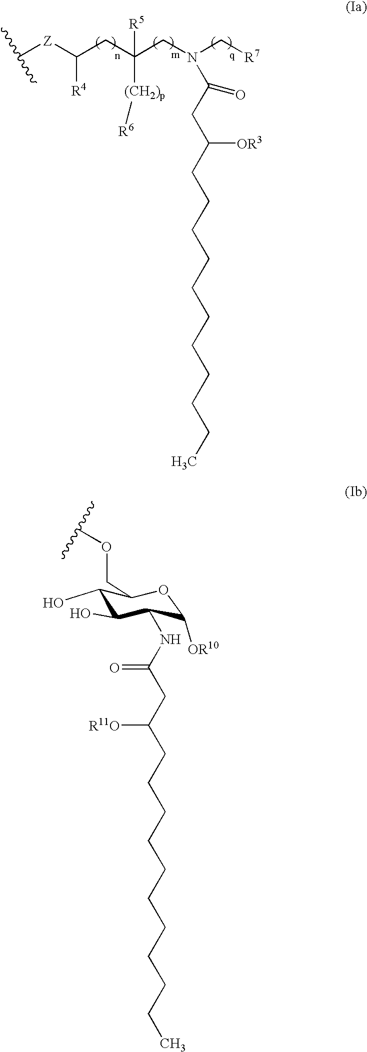 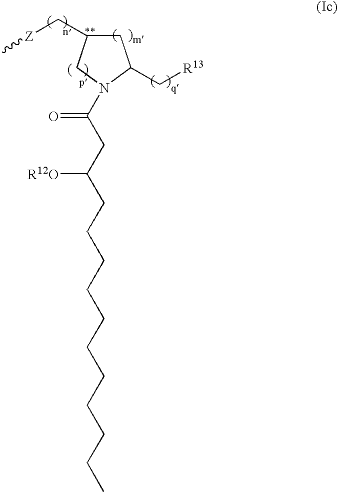
wherein the subscripts n, m, p, q, n′, m′, p′ and q′ are each independently an integer of from 0 to 6, provided that the sum of p′ and m′ is an integer from 0 to 6; R³, R¹¹, and R¹² are independently a saturated or unsaturated optionally substituted aliphatic (C₂-C₂₄) acyl group, provided that when X is formula (Ia), one of R¹, R² and R³ is optionally hydrogen; R⁴ and R⁵ are independently selected from H and methyl; R⁶ and R⁷ are independently selected from H, OH, (C₁-C₄) oxyaliphatic groups, —PO₃H₂, —OPO₃H₂, —SO₃H, —OSO₃H, —NR¹⁵R¹⁶, —SR¹⁵, —CN, —NO₂, —CHO, —CO₂R¹⁵, —CONR¹⁵R¹⁶, —PO₃R¹⁵R¹⁶, —OPO₃R¹⁵R¹⁶, —SO₃R¹⁵ and —OSO₃R¹⁵, wherein R¹⁵ and R¹⁶ are each independently selected from H and (C₁-C₄) aliphatic groups; R¹⁰ is selected from H, CH₃, —PO₃H₂, ω-phosphonooxy(C₂-C₂₄)alkyl, and ω-carboxy(C₁-C₂₄)alkyl; R¹³ is independently selected from H, OH, (C₁-C₄) oxyaliphatic groups, —PO₃R¹⁷R¹⁸, —OPO₃R¹⁷R¹⁸, —SO₃R¹⁷, —OSO₃R¹⁷, —NR⁷R¹⁸, —SR¹⁷, —CN, —NO₂, —CHO, —CO₂R⁷, and —CONR¹⁷R¹⁸, wherein R¹⁷ and R¹⁸ are each independently selected from H and (C₁-C₄) aliphatic groups; and Z is —O— or —S—.

10. The composition of claim 8 wherein the aminoalkyl glucosaminide phosphate has the formula: 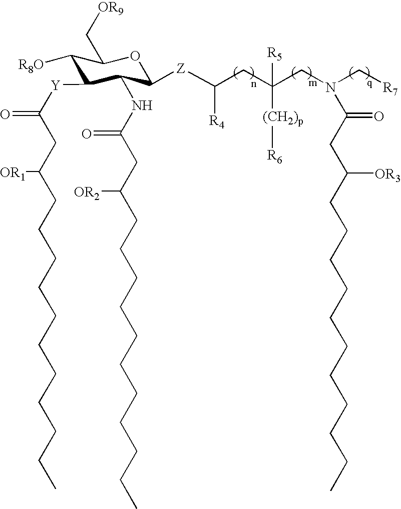
and pharmaceutically acceptable salts, derivatives and biologically active fragments thereof, wherein Z represents an oxygen or sulfur atom, Y represents an oxygen atom or NH group, “n”, “m”, “p” and “q” are integers independently selected from 0 to 6, R₁, R₂, and R₃ represent fatty acyl residues, including saturated, unsaturated, and branched acyl groups, having 6 to 16 carbon atoms, R₄ and R₅ are independently selected from hydrogen and methyl, R₆ and R₇ are independently selected from hydrogen, hydroxy, alkoxy, phosphono, phosphonooxy, sulfo, sulfooxy, amino, mercapto, cyano, nitro, formyl or carboxy and esters and amides thereof; R₈ and R₉ are independently selected from phosphono or hydrogen, wherein at least one of R₈ and R₉ is phosphono.

11. The composition of claim 8 wherein the aminoalkyl glucosaminide phosphate has the formula: 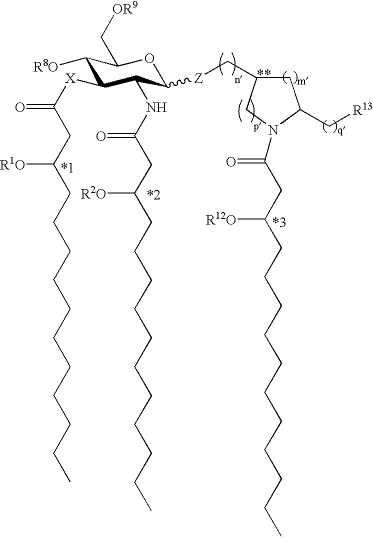
and pharmaceutically acceptable salts thereof, wherein Z is a member selected from the group consisting of —O— and —NH—; Y is a member selected from the group consisting of —O— and —S—; R¹, R² and R³ are each members independently selected from the group consisting of (C₂-C₂₄) acyl; R⁴ is a member selected from the group consisting of —H and —PO₃R⁷R⁸, wherein R⁷ and R⁸ are each members independently selected from the group consisting of —H and (C₁-C₄)alkyl; R⁵ is a member selected from the group consisting of —H, —CH₃ and —PO₃R⁹R¹⁰, wherein R⁹ and R¹⁰ are each members independently selected from the group consisting of —H and (C₁-C₄)alkyl; R⁶ is selected from H, OH, (C₁-C₄)alkoxy, —PO₃R¹¹R¹², —OPO₃R¹¹R¹², —SO₃R¹¹, —OSO₃R¹¹, —NR¹¹R¹², —SR¹¹, —CN, —NO₂, —CHO, —CO₂R¹¹, and —CONR¹¹R¹², wherein R¹¹ and R¹² are each independently selected from H and (C—C₄)alkyl, with the provisos that one of R⁴ and R⁵ is a phosphorus-containing group and that when R⁴ is —PO₃R⁷R⁸, R⁵ is other than —PO₃R⁹R¹⁰; wherein “*1”, “*2”, “*3” and “**” represent chiral centers; wherein the subscripts n′, m′, p′ and q′ are each independently an integer from 0 to 6, with the proviso that the sum of p′ and m′ is from 0 to 6.

12. The composition of claim 9 wherein X is formula (Ia); Y is oxygen; Z is oxygen; R₁, R₂ and R₃ are all aliphatic acyl groups, and R₈ is a phosphorus-containing group;

13. The composition of claim 12 wherein n and m are both 0.

14. The composition of claim 12 wherein R₆ is COOH

15. The composition of claim 13 wherein R6 is COOH.

16. The composition of claim 9 wherein X is formula (Ia); Y is oxygen; n, m, p, and q are each 0; R₄, R₅, R₇ and R₉ are each H, and R₈ is a phosphorous-containing group.

17. The composition of claim 16 wherein R₁, R₂ and R₃ are all aliphatic acyl groups

18. The composition of claim 17 in which R₁, R₂ and R₃ are each n-C₁₃H₂₇CO and R₆ is hydrogen.

19. The composition of claim 17 in which R₁, R₂ and R₃ are each n-C₉H₁₉C0 and R₆ is hydrogen.

20. The composition of claim 17 in which R₁ is n-C₉H₁₉CO, R₂ and R₃ are each n-C₅H₁₁CO, and R₆ is COOH.

21. The composition of claim 17 in which R₁ and R₃ are each n-C₅H₁₁CO, R₂ is n-C₉H₁₉CO and R₆ is COOH.

22. The composition of claim 6 wherein said adjuvant comprises a saponin.

23. The composition of claim 22 wherein the saponin is selected from naturally obtained saponins, synthetically obtained saponins, saponin conjugates, saponin derivatives and saponin mimetics.

24. The composition of claim 22 wherein the saponin is selected from Quillaja saponins, triterpene saponin-lipophile conjugates, saponin/antigen covalent conjugates, and compounds having the formula: 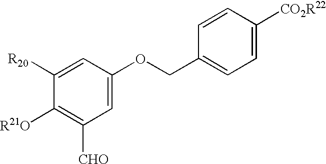
wherein, R₂₀ is hydrogen or —C(O)H; R²¹ is a member selected from the group consisting of hydrogen, an optionally substituted C₁₋₂₀ aliphatic group, a saccharyl group, and a group represented by the formula —C(O)-[C(R²³)(R²⁴)]ₖ—COOH, wherein each R²³ and R²⁴ independently is a member selected from the group consisting of hydrogen and optionally substituted C₁₋₁₀ aliphatic groups, and k is a number from 1 to 5; R²² is a member selected from the group consisting of hydrogen, an optionally substituted C₁₋₂₀ aliphatic group, and a group represented by the formula —(CH₂)ᵣCH(OH)(CH₂)ₜOR²⁵, wherein r and t are independently 1 or 2, and R²⁵ is an optionally substituted C₂₋₂₀ aliphatic group, or a group represented by the formula 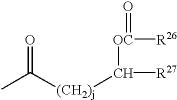
wherein j is 1-5, and R²⁶ and R²⁷ are independently selected from the group consisting of hydrogen and optionally substituted C₁₋₂₀ aliphatic groups; or a pharmacologically acceptable salt thereof.

25. The composition of claim 22 wherein the saponin is QS-21.

26. The composition of claim 6 wherein said adjuvant comprises a mixture of an aminoalkyl glucosaminide phosphate and a saponin.

27. The composition of claim 26 wherein the saponin is QS-21

28. The composition of claim 26 wherein the aminoalkyl glucosaminide phosphate has the formula 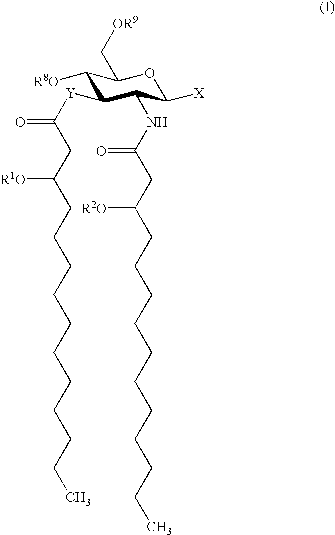
and pharmaceutically acceptable salts and derivatives thereof, wherein Y is —O— or —NH—; R¹ and R² are each independently selected from saturated and unsaturated (C₂-C₂₄) aliphatic acyl groups; R⁸ is —H or —PO₃R¹¹R¹², wherein R¹¹ and R¹² are each independently —H or (C₁-C₄) aliphatic groups; R⁹ is —H, —CH₃ or —PO₃R¹³R¹⁴, wherein R¹³ and R¹⁴ are each independently selected from —H and (C₁-C₄) aliphatic groups; and wherein at least one of R⁸ and R⁹ is a phosphorus-containing group, but R⁸ and R⁹ are not both phosphorus-containing groups; and X is a group selected from the formulae: 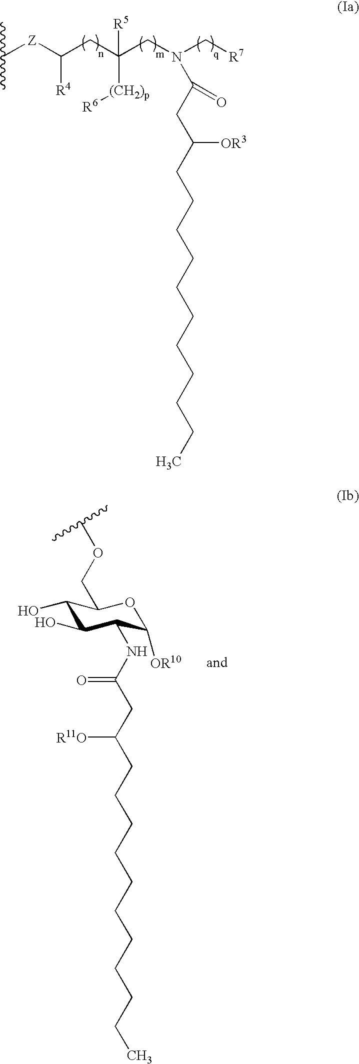 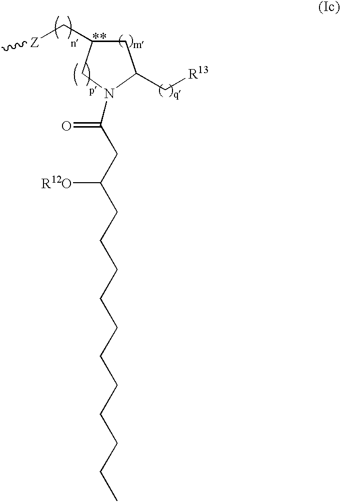
wherein the subscripts n, m, p, q, n′, m′, p′ and q′ are each independently an integer of from 0 to 6, provided that the sum of p′ and m′ is an integer from 0 to 6; R³, R¹¹, and R¹² are independently a saturated or unsaturated optionally substituted aliphatic (C₂-C₂₄) acyl group, provided that when X is formula (Ia), one of R¹, R² and R³ is optionally hydrogen; R⁴ and R⁵ are independently selected from H and methyl; R⁶ and R⁷ are independently selected from H, OH, (C₁-C₄) oxyaliphatic groups, —PO₃H₂, —OPO₃H₂, —SO₃H, —OSO₃H, —NR¹⁵R⁶, —SR⁵, —CN, —NO₂, —CHO, —CO₂R⁵, —CONR¹⁵R¹⁶, —PO₃R¹⁵R¹⁶, —OPO₃R¹⁵R¹⁶, —SO₃R¹⁵ and —OSO₃R¹⁵, wherein R¹⁵ and R¹⁶ are each independently selected from H and (C₁-C₄) aliphatic groups; R¹⁰ is selected from H, CH₃, —PO₃H₂, ω-phosphonooxy(C₂-C₂₄)alkyl, and ω-carboxy(C₁-C₂₄)alkyl; R¹³ is independently selected from H, OH, (C₁-C₄) oxyaliphatic groups, —PO₃R¹⁷R¹⁸, —OPO₃R¹⁷R¹⁸, —SO₃R¹⁷, —OSO₃R¹⁷, —NR¹⁷R¹⁸, —SR¹⁷, —CN, —NO₂, —CHO, —CO₂R¹⁷, and —CONR¹⁷R¹⁸, wherein R¹⁷ and R¹⁸ are each independently selected from H and (C₁-C₄) aliphatic groups; and Z is —O— or —S—.

29. The composition of claim 6 wherein said adjuvant induces an immune response predominantly of the Th1 type.

30. The composition of claim 29 wherein said adjuvant induces a cytokine selected from the group consisting of IFN-γ, TNFα, IL-2 and IL-12.

31. A pharmaceutical composition comprising the composition of claim 1 and a pharmaceutically acceptable carrier.

32 A method for enhancing an immune response to a polypeptide of interest comprising the step of immunizing a mammal with the composition of claim 1.

33. A method for reducing a neutralizing antibody response to a recombinant virus comprising the step of immunizing a mammal with the composition of claim 1.
